(19) United States
(12) Patent Application Publication
(10) Pub. No.: US 2021/0186984 A1
(43) Pub. Date: Jun. 24, 2021

(54) CERTAIN (2S)-N-[(1S)-1-CYANO-2-PHENYLETHYL]-1,4-OXAZEPANE-2-CARBOXAMIDES FOR TREATING ANCA ASSOCIATED VASCULITIDES

(71) Applicant: Insmed Incorporated, Bridgewater, NJ (US)

(72) Inventors: Keith DIPETRILLO, Bridgewater, NJ (US); Carlos FERNANDEZ, Bridgewater, NJ (US); Jimin ZHANG, Bridgewater, NJ (US)

(21) Appl. No.: 17/157,216

(22) Filed: Jan. 25, 2021

Related U.S. Application Data
(63) Continuation of application No. 16/269,191, filed on Feb. 6, 2019, now abandoned.
(60) Provisional application No. 62/627,408, filed on Feb. 7, 2018.
(60) Provisional application No. 62/694,318, filed on Jul. 5, 2018.
(60) Provisional application No. 62/772,956, filed on Nov. 29, 2018.

(51) Int. Cl. A61K 31/553; C07K 16/28; C07K 16/24; A61K 31/675; A61P 7/00; A61K 31/573; A61K 45/06; C07C 15/00

(52) U.S. Cl. CPC A61K 31/553; C07K 16/2887; C07K 16/241; A61K 31/675; A61K 9/0053; A61K 31/573; A61K 45/06; C07C 15/00; A61P 7/00

(57) ABSTRACT

The present disclosure relates to methods for treating an ANCA associated vasculitis, for example, granulomatosis with polyangiitis (GPA), with compositions comprising an effective amount of certain (2S)-N-[(1S)-1-cyano-2-phenylethyl]-1,4-oxazepane-2-carboxamide compounds of Formula (I), including pharmaceutically acceptable salts thereof,

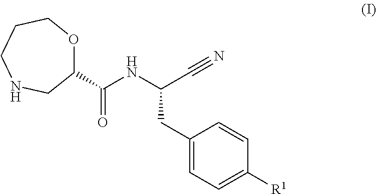

that inhibit dipeptidyl peptidase 1 (DPP1) activity. In one embodiment, the compound of Formula (I) is (2S)-N-{(1S)-1-cyano-2-[4-(3-methyl-2-oxo-2,3-dihydro-1,3-benzoxazol-5-yl)phenyl]ethyl}-1,4-oxazepane-2-carboxamide (INS1007).

### CROSS-REFERENCE TO RELATED APPLICATIONS

This application claims priority from U.S. Provisional Patent Application No. 62/627,408, filed Feb. 7, 2018; U.S. Provisional Patent Application No. 62/694,318, filed Jul. 5, 2018; and U.S. Provisional Patent Application No. 62/772,956, filed Nov. 29, 2018, the disclosure of each of which is incorporated by reference herein in their entireties.

### BACKGROUND OF THE INVENTION

Granulomatosis with polyangiitis (GPA) (formerly known as Wegener' s disease or Wegener's granulomatosis), microscopic polyangiitis (MPA), idiopathic crescentic granulonephritis and Churg-Strauss syndrome are disorders associated with vasculitis that is characterized by inflammation of the blood vessels and the presence of circulating antineutrophil cytoplasmic autoantibodies (ANCA). For GPA patients, the ANCAs are mainly directed against proteinase 3 (PR3) (Pagnoux and Guillevin (2015). Expert Rev. Clin. Immunol. 11(3), pp. 339-348). For MPA patients, the ANCAs are mainly directed against myeloperoxidase (MPO). However, ANCAs against PR3 have also been reported in MPA patients. Schirmer et al. (2016). Rheumatology 55, pp. 71-719.

PR3 is not only stored in azurophilic granules, released during neutrophil activation; but also exists in a membrane bound form (mbPR3) (Korkmaz et al. (2013). Semin. Immunopathol. 35, pp. 411-421). mbPR3 is the prime antigenic target of ANCA in GPA patients. ANCA binding to mbPR3 results in excessive activation of neutrophils, which produces reactive oxygen species, liberation of granular proteinases and necrosis of surrounding tissue (Korkmaz et al. (2013). Semin. Immunopathol. 35, pp. 411-421).

Although GPA can affect any organ, it often results in necrotizing granulomatous inflammation and vasculitis of small vessels in the lower respiratory track and kidneys (Cartin-Ceba et al. (2012). Arthritis & Rheumatism 64(11), pp. 3770-3778; Korkmaz et al. (2013). Semin. Immunopathol. 35, pp. 411-421). GPA affects about 1 in 20,000 people and while it can occur at any age, it most often presents in the fourth and fifth decades of life (Korkmaz et al. (2013). Semin. Immunopathol. 35, pp. 411-421). MPA is characterized histologically by the involvement of small vessels such as capillaries, venules and/or arterioles, and the absence of granuloma. Guilleven et al. (1999). Arthritis & Rheumatism 42(3), pp. 421-430.

The present disclosure addresses the need for a therapy effective for the treatment of ANCA associated vasculitides, including GPA and MPA.

### SUMMARY OF THE INVENTION

In one aspect, a method is provided for treating an ANCA associated vasculitis in a patient in need thereof. The method comprises, in one embodiment, administering to the patient, a pharmaceutical composition comprising an effective amount of a compound of formula (I), or a pharmaceutically acceptable salt thereof:

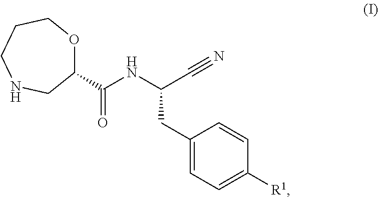

wherein,
R¹ is

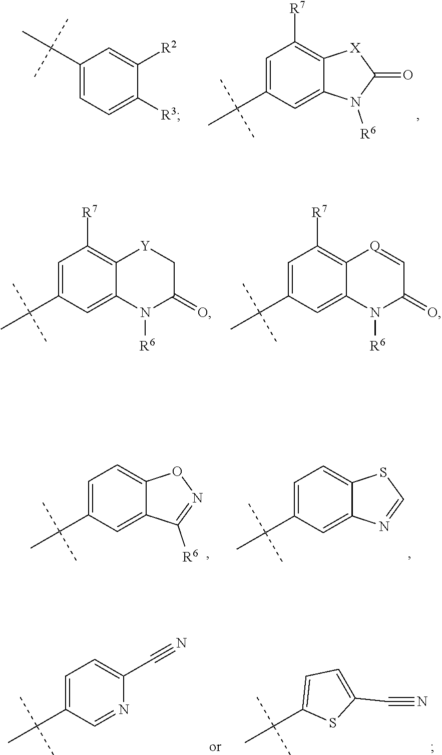

R² is hydrogen, F, Cl, Br, OSO₂C₁₋₃alkyl, or Cl₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring; or
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

The ANCA associated vasculitis in one embodiment, is GPA.

In another embodiment, the ANCA associated vasculitis is MPA.

In one embodiment of the methods provided herein, the pharmaceutical composition comprises an effective amount of (2S)-N-{(1S)-1-cyano-2-[4-(3-methyl-2-oxo-2,3-dihydro-1,3-benzoxazol-5-yl)phenyl]ethyl}-1,4-oxazepane-2-carboxamide, (also referred to herein as INS 1007),

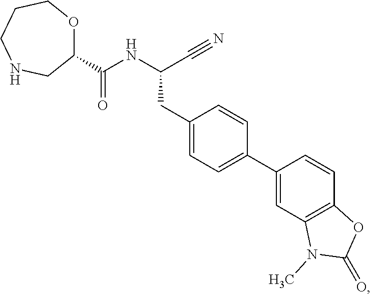 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment, the patient has an active ANCA associated vasculitis (e.g., active GPA or MPA). In another embodiment, the patient is in remission of an ANCA associated vasculitis (e.g., in remission of GPA or MPA). In a further embodiment, the ANCA associated vasculitis is GPA.

In one embodiment, the patient has never been treated with other ANCA associated vasculitis drugs prior to the administration of a pharmaceutical composition of the present disclosure. In another embodiment, the patient is treated with one or more other ANCA associated vasculitis drugs, such as an anti-CD20 monoclonal antibody (e.g., rituximab), an anti-TNF-a monoclonal antibody (e.g., infliximab), cyclophosphamide, or a steroid (e.g., a corticosteroid, such as a glucocorticoid), prior to the administration of the pharmaceutical compositions of the present disclosure, and optionally treated with one or more of these drugs during and/or after the administration of the pharmaceutical compositions of the present disclosure.

Administration routes include oral administration. Administration schedules can be determined by the user of the method, e.g., a prescribing physician. In one embodiment, administration is once daily. In another embodiment, administration is twice daily. In another embodiment, administration 1× daily, once every other day, once every third day, once every fourth day, 2× weekly, 3× weekly or 4× weekly.

The present disclosure also provides the aforementioned pharmaceutical compounds and compositions comprising the same for use in treating the conditions in a patient in need thereof in accordance with a treatment method described herein, as well as the use of the aforementioned compounds in the manufacture of medicaments for treating the conditions in a patient in need thereof in assccordance with a treatment method described herein.

The present disclosure further provides kits and articles of manufacture to be used in accordance with a treatment method described herein. In some embodiments, the article of manufacture or kit comprises a container containing the pharmaceutical composition and a label associated with the container. The container may contain single or multiple, e.g., 10, 20 or 30, daily doses of the pharmaceutical composition. The container may also contain one or more other ANCA associated vasculitis drugs, such as those described above.

### BRIEF DESCRIPTION OF THE FIGURES

FIG. 1 is a graph of surface proteinase 3 (PR3) expression (% of untreated cells, mean fluorescence intensity (MFI)) in neutrophils derived from either bone marrow (BM) or umbilical cord blood (UC) stem cells as a function of INS1007 concentration. INS1007 was added in the cell culture medium during neutrophil differentiation. *P<0.05 vs. the lowest INS 1007 concentration.

FIG. 2 is a graph of surface proteinase 3 (PR3) expression (% positive cells) in neutrophils derived from either bone marrow (BM) or umbilical cord blood (UC) stem cells as a function of INS1007 concentration. INS 1007 was added in the cell culture medium during neutrophil differentiation. *P<0.05 vs. untreated cells.

FIG. 3 is a graph of total proteinase 3 (PR3) expression (surface and intracellular, % of untreated) in neutrophils derived from either bone marrow stem cells (BM) or umbilical cord blood stem cells (UC) as a function of INS 1007 concentration. INS 1007 was added in the cell culture medium during neutrophil differentiation. *P<0.05 vs. the lowest INS1007 concentration.

FIG. 4 is a graph of the percentages of neutrophils that showed positive total PR3 expression as a function of INS1007 concentration. Neutrophils were derived from either bone marrow stem cells (BM) or umbilical cord blood stem cells (UC).

FIG. 5 is a graph of neutrophil elastase (NE) activity (% of untreated control) in neutrophils derived from either bone marrow or umbilical cord blood stem cells as a function of INS1007 concentration. INS 1007 was added in the cell culture medium during neutrophil differentiation. *P<0.05 vs. the lowest INS1007 concentration.

FIG. 6 is a graph of proteinase 3 (PR3) activity (% of untreated control) in neutrophils derived from either bone marrow or umbilical cord blood stem cells as a function of INS1007 concentration. INS1007 was added in the cell culture medium during neutrophil differentiation. *P<0.05 vs. the lowest INS1007 concentration.

FIG. 7 is a graph of neutrophil elastase (NE) activity (ng/μg of total protein) in C57BL/6 mice as a function of daily INS1007 oral dose.

### DETAILED DESCRIPTION OF THE INVENTION

GPA is a rare systemic autoimmune necrotizing granulomatous inflammation and systemic vasculitis of small blood vessels (Pagnoux (2016). Eur J Rheumatol. 3(3), pp. 122-33; Schonermarck et al. (2015). Nephrol Dial Transplant. 2015; 30(Supp11): i46-52, each incorporated by reference here in its entirety for all purposes). It most commonly affects the upper and/or lower respiratory tracts, kidneys, skin, and peripheral nerves. The pathogenesis of GPA involves neutrophils and ANCAs. In most patients with GPA, ANCAs bind to membrane-bound PR3 (mPR3), triggering neutrophil activation with the release of ROS and proteases, including active NSPs (PR3, NE, and Cat G). This extracellular proteolytic activity contributes to the vascular necrosis of endothelial cells observed in GPA patients (Jerke et al. (2015). Kidney Int. 88(4):764-775; Kettritz (2016). Immunol Rev. 273(1): 232-248, each incorporated by reference here in its entirety for all purposes).

Left untreated, GPA is a severe and progressive disease that leads to death from multisystem organ failure (Millet et al. (2013). Ann Rheum Dis. 72(8):1273-9; Yates and Watts (2017). Clin Med (Lond). 17(1):60-64, each incorporated by reference here in its entirety for all purposes). The standard therapy includes cytotoxic immunosuppressive agents such as cyclophosphamide, and most recently rituximab, combined with high-dose glucocorticoids. Despite treatment, disease relapses are common (Guillevin et al. (2014). N Engl J Med. 371(19): 1771-80; Jayne et al. (2003). N Engl J Med. 2003; 349(1):36-44; Pagnoux et al. (2008). N Engl J Med. 359(26):2790-2803, each incorporated by reference herein in their entireties for all purposes) and the mortality rates remain high (Heijl et al. (2017). RMD open; Volume 3, Issue 1, p:e000435; Pearce et al. (2017). Rheumatology (Oxford). 56(4):589-96, each incorporated by reference herein in their entireties for all purposes). Furthermore, there is a narrow safety margin of current therapies due to systemic toxicities. Taken together, there remains a high unmet need for adequate treatment of GPA, particularly for the maintenance of remission. This residual unmet need underscores the necessity to develop novel therapeutic strategies, such as may be achieved by the methods of the present invention.

The methods provided herein employ reversible inhibitors of the the lysosomal cysteine protease dipeptidyl peptidase 1 (DPP1) in methods for treating an ANCA associated vasculitis, such as, but not limited to, GPA and/or MPA. Without wishing to be bound by theory, it is thought that the compounds of formula (I), administered via the methods provided herein have beneficial effects via inhibition of PR3 activation via the upstream inhibition of DPP1. Inhibition of DPP1, without wishing to be bound by theory, reduces the amount of activated NSPs available for release during neutrophil degranulation. Moreover, inhibition of PR3 activation in turn leads to lack of PR3 interaction and expression at the neutrophil membrane surface (mPR3). The decreased mPR3 in turn, may limit the targets to which PR3-specific ANCA can bind, and in turn, attenuates neutrophil activation. Moreover, without wishing to be bound by theory, because autoantibodies in patients with MPA bind to surface PR3 and/or to stimulate NSP release and subsequent tissue damage, the pharmacological effects of INS1007 on neutrophils may treat MPA and other myeloperoxidase (MPO)-ANCA related disorders by reducing activities of NSP to decrease the tissue damage following neutrophil degranulation.

Neutrophils contain four main types of granules: (i) azurophilic or primary granules, (ii) specific or secondary granules, (iii) gelatinase or tertiary granules, and (iv) secretory granules. Azurophilic granules are believed to be the first to form during neutrophil maturation in the bone marrow and are characterized by the expression of related neutrophil serine proteases (NSPs): neutrophil elastase (NE), proteinase 3 (PR3), and cathepsin G (CatG). DPP1 is the proteinase that activates these three NSPs by removal of the N-terminal dipeptide sequences from their precursors during azurophilic granule assembly (Pham et al. (2004). J Immunol. 173(12), pp. 7277-7281). DPP1 is broadly expressed in tissues but is highly expressed in cells of hematopoietic lineage such as neutrophils.

The three NSPs, abundantly secreted into the extracellular environment upon neutrophil activation at inflammatory sites, are thought to act in combination with reactive oxygen species to assist in degradation of engulfed microorganisms inside phagolysosomes. A fraction of the released proteases remains bound in an active form on the external surface of the plasma membrane so that both soluble and membrane-bound NSPs can regulate the activities of a variety of biomolecules such as chemokines, cytokines, growth factors, and cell surface receptors. Regulation is thought to occur by either converting the respective biomolecule to an active form or by degrading the biomolecule by proteolytic cleavage. Secreted proteases can stimulate mucus secretion and inhibit mucociliary clearance, but also activate lymphocytes and cleave apoptotic and adhesion molecules (Bank and Ansorge (2001). J Leukoc Biol. 69, pp. 197-206; Pham (2006). Nat Rev Immunol. 6, pp. 541-550; Meyer-Hoffert (2009). Front Biosci. 14, pp. 3409-3418; Voynow et al. (2004). Am J Physiol Lung Cell Mol Physiol. 287, pp. L1293-302; the disclosure of each of which is incorporated by reference in its entirety for all purposes).

As used herein, “C1-3” means a carbon group having 1,2 or 3 carbon atoms.

The term “alkyl”, unless otherwise noted, includes both straight and branched chain alkyl groups and may be substituted or non-substituted. “Alkyl” groups include, but are not limited to, methyl, ethyl, n-propyl, i-propyl, butyl, pentyl. Exemplary C₁₋₃alkyl groups include methyl, ethyl, and propyl, which includes (e.g., n-propyl, i-propyl).

Substituted alkyl refers to an alkyl group as defined above, having from 1 to 8 substituents, e.g., from 1 to 3 substituents or from 1 to 5 substituents. Exemplary substitutents include alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclic, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, and —SO₂-heteroaryl.

The term “pharmaceutically acceptable”, unless otherwised noted, is used to characterize a moiety (e.g., a salt, dosage form, or excipient) as being appropriate for use in accordance with sound medical judgment. In general, a pharmaceutically acceptable moiety has one or more benefits that outweigh any deleterious effect that the moiety may have. Deleterious effects may include, for example, excessive toxicity, irritation, allergic response, and other problems and complications.

Provided herein are methods for treating an ANCA associated vasculitis (e.g., GPA or MPA) via administration of a pharmaceutical composition comprising an effective amount of a compound of formula (I), or a pharmaceutically acceptable salt thereof:

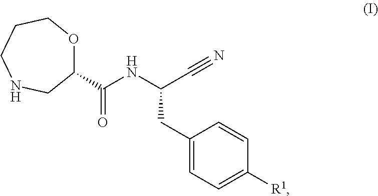

wherein,
R¹ is

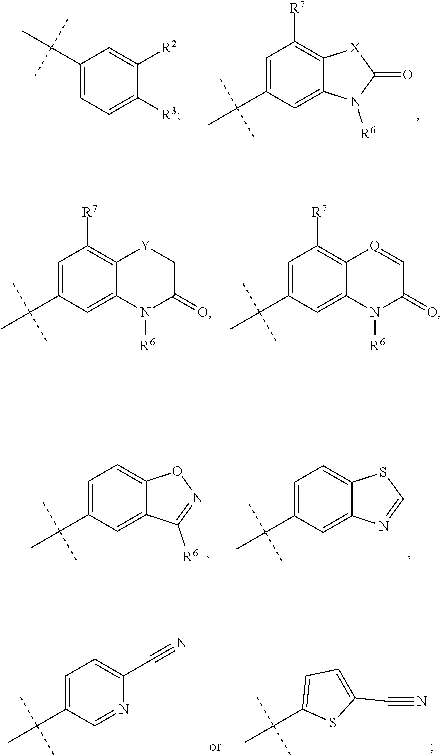

R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring; or
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

In one embodiment R¹ is

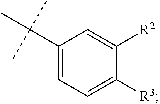

R² is hydrogen, F, Cl, Br, OSO₂C₁₋₃alkyl, or C₁₋₃alkyl; R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring.

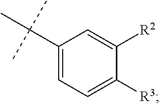

In a further embodiment, R¹ is R² is hydrogen, F, Cl or C₁₋₃alkyl; and R³ is hydrogen, F, Cl, CN or SO₂C₁₋₃alkyl.

In still a further embodiment, R¹ is

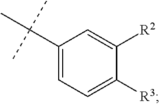

R² is hydrogen, F or C₁₋₃alkyl; and R³ is hydrogen, F or CN.

In another embodiment, R¹ is

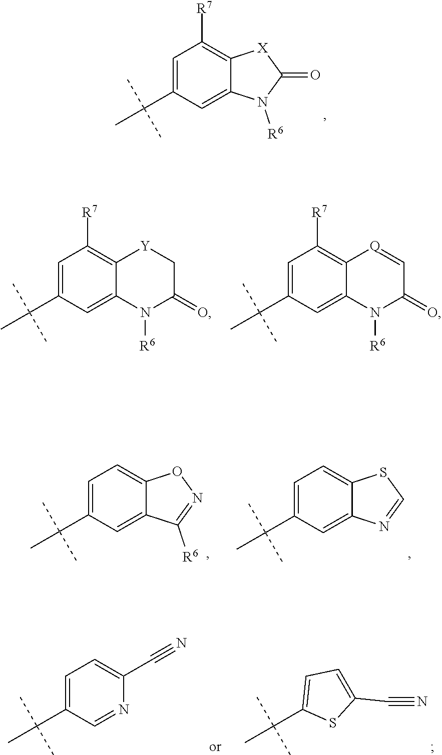

X is O, S or CF₂; Y is O or S; Q is CH or N; R⁶ is C₁₋₃alkyl, wherein the C₁₋₃alkyl is optionally substituted by 1, 2 or 3 F and/or optionally substituted by OH, OC₁₋₃alkyl, N(C₁₋₃alkyl)₂, cyclopropyl, or tetrahydropyran; and R⁷ is hydrogen, F, Cl or CH₃.

In still a further embodiment, R¹ is

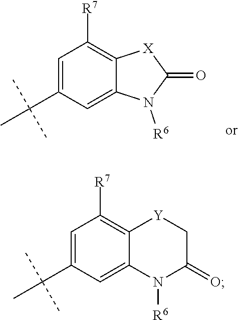

X is O, S or CF₂; Y is O or S; R⁶ is C₁₋₃alkyl, optionally substituted by 1, 2 or 3 F and optionally substituted by OH, OC₁₋₃alkyl, N(C₁₋₃alkyl)₂, cyclopropyl, or tetrahydropyran; and R⁷ is hydrogen, F, Cl or CH₃.

In still a further embodiment, R¹ is

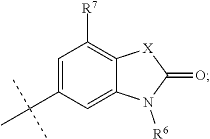

X is O, S or CF₂; R⁶ is C₁₋₃alkyl, wherein the C₁₋₃alkyl is optionally substituted by 1, 2 or 3 F; and R⁷ is hydrogen, F, Cl or CH₃.

In still a further embodiment, R¹ is

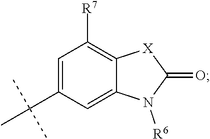

X is O; R⁶ is C₁₋₃alkyl, wherein the C₁₋₃alkyl is optionally substituted by 1, 2 or 3 F; and R⁷ is hydrogen. In a further embodiment, R⁶ is methyl or ethyl.

In another embodiment, R¹ is

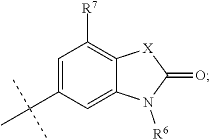

X is O; R⁶ is C₁₋₃alkyl, wherein the C₁₋₃alkyl is optionally substituted by 1, 2 or 3 F; and R⁷ is hydrogen. In a further embodiment, R⁶ is ethyl.

In yet another embodiment, R¹ is

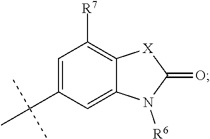

X is O; R⁶ is C₁₋₃alkyl, wherein the C₁₋₃alkyl is optionally substituted by 1, 2 or 3 F; and R⁷ is hydrogen. In a further embodiment, R⁶ is methyl.

In one embodiment, R² is hydrogen, F, Cl, Br, OSO₂C₁₋₃alkyl or C₁₋₃alkyl.

In a further embodiment, R² is hydrogen, F, Cl or C₁₋₃alkyl.

In still a further embodiment, R² is hydrogen, F or C₁₋₃alkyl.

In one embodiment, R³ is hydrogen, F, Cl, Br, CN, CF₃, SO₂C₁₋₃alkyl CONH₂ or SO₂NR⁴R⁵, wherein R⁴ and R⁵ together with the nitrogen atom to which they are attached form an azetidine, pyrrolidine or piperidine ring.

In a further embodiment, R³ is selected from hydrogen, F, Cl, CN or SO₂C₁₋₃alkyl.

In still a further embodiment, R³ is selected from hydrogen, F or CN.

In one embodiment, R⁶ is C₁₋₃alkyl, wherein said C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran.

In a further embodiment, R⁶ is C₁₋₃alkyl, wherein said C₁₋₃alkyl is optionally substituted by 1, 2 or 3 F. In still a further embodiment, R⁶ is methyl or ethyl. In still a further embodiment, R⁶ is methyl.

In one embodiment, R⁷ is hydrogen, F, Cl or CH₃. In a further embodiment R⁷ is hydrogen.

In one embodiment of the methods provided herein, the composition administered to the patient comprises an effective amount of (2S)-N-{(1S)-1-cyano-2-[4-(3-methyl-2-oxo-2,3-dihydro-1,3-benzoxazol-5-yl)phenyl]ethyl}-1,4-oxazepane-2-carboxamide (INS1007):

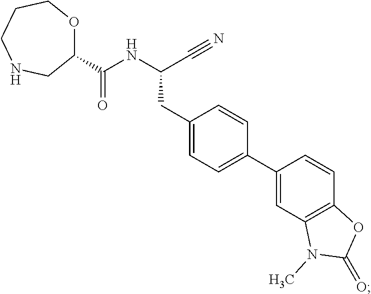 [SMILES: Cn1c(=O)oc2ccc(-c3ccc(C[C@@H](C#N)NC(=O)[C@@H]4CNCCCO4)cc3)cc21]

or a pharmaceutically acceptable salt thereof.

In one embodiment, the compound of formula (I) is:

(2S)-N-[(1S)-1-Cyano-2-(4′-cyanobiphenyl-4-yl)ethyl]-1,4-oxazepane-2-carboxamide,

(2S)-N-{(1S)-1-Cyano-2-[4-(3-methyl-2-oxo-2,3-dihydro-1,3-benzoxazol-5-yl)phenyl]ethyl}-1,4-oxazepane-2-carboxamide,

(2S)-N-{(1S)-1-Cyano-2-[4-(3,7-dimethyl-2-oxo-2,3-dihydro-1,3-benzoxazol-5-yl)phenyl]ethyl}-1,4-oxazepane-2-carboxamide,

4′-[(2S)-2-Cyano-2-{[(2S)-1,4-oxazepan-2-ylcarbonyl]amino}ethyl]biphenyl-3-yl methanesulfonate,

(2S)-N-{(1S)-1-Cyano-2-[4-(3-methyl-1,2-benzoxazol-5-yl)phenyl]ethyl}-1,4-oxazepane-2-carboxamide,

(2S)-N-{(1S)-1-Cyano-2-[4′-(trifluoromethyl)biphenyl-4-yl]ethyl}-1,4-oxazepane-2-carboxamide,

(2S)-N-[(1S)-1-Cyano-2-(3′,4′-difluorobiphenyl-4-yl)ethyl]-1,4-oxazepane-2-carboxamide,

(2S)-N-{(1S)-1-Cyano-2-[4-(6-cyanopyridin-3-yl)phenyl]ethyl}-1,4-oxazepane-2-carboxamide,

(2S)-N-{(1S)-1-Cyano-2-[4-(4-methyl-3-oxo-3,4-dihydro-2H-1,4-benzothiazin-6-yl)phenyl]ethyl}-1,4-oxazepane-2-carboxamide,

(2S)-N-{(1S)-1-Cyano-2-[4-(3-ethyl-7-methyl-2-oxo-2,3-dihydro-1,3-benzoxazol-5-yl)phenyl]ethyl}-1,4-oxazepane-2-carboxamide,

(2S)-N-[(1S)-1-Cyano-2-{4-[3-(2-hydroxy-2-methylpropyl)-2-oxo-2,3-dihydro-1,3-benzoxazol-5-yl]phenyl}ethyl]-1,4-oxazepane-2-carboxamide,

(2S)-N-[(1S)-1-Cyano-2-{4-[3-(2,2-difluoroethyl)-7-fluoro-2-oxo-2,3-dihydro-1,3-benzoxazol-5-yl]phenyl}ethyl]-1,4-oxazepane-2-carboxamide,

(2S)-N-[(1S)-1-Cyano-2-(4-{3-[2-(dimethylamino)ethyl]-2-oxo-2,3-dihydro-1,3-benzoxazol-5-yl}phenyl)ethyl]-1,4-oxazepane-2-carboxamide,

(2S)-N-{(1S)-1-Cyano-2-[4-(3,3-difluoro-1-methyl-2-oxo-2,3-dihydro-1H-indol-6-yl)phenyl]ethyl}-1,4-oxazepane-2-carboxamide,

(2S)-N-{(1S)-1-Cyano-2-[4-(7-fluoro-3-methyl-2-oxo-2,3-dihydro-1,3-benzoxazol-5-yl)phenyl]ethyl}-1,4-oxazepane-2-carboxamide,

(2S)-N-{(1S)-1-Cyano-2[4-(3-ethyl-2-oxo-2,3-dihydro-1,3-benzoxazol-5-yl)phenyl]ethyl}-1,4-oxazepane-2-carboxamide,

(2S)-N-[(1S)-1-Cyano-2-{4-[3-(cyclopropylmethyl)-2-oxo-2,3-dihydro-1,3-benzoxazol-5-yl]phenyl}ethyl]-1,4-oxazepane-2-carboxamide,

(2S)-N-[(1S)-1-Cyano-2-{4-[3-(2-methoxyethyl)-2-oxo-2,3-dihydro-1,3-benzothiazol-5-yl]phenyl}ethyl]-1,4-oxazepane-2-carboxamide,

(2S)-N-[(1S)-1-Cyano-2-{4-[2-oxo-3-(propan-2-yl)-2,3-dihydro-1,3-benzoxazol-5-yl]phenyl}ethyl]-1,4-oxazepane-2-carboxamide,

(2S)-N-[(1S)-1-Cyano-2-{4-(4-methyl-3-oxo-3,4-dihydro-2H-1,4-benzoxazin-6-yl)phenyl]ethyl}-1,4-oxazepane-2-carboxamide,

(2S)-N-[(1S)-1-Cyano-2-{4-[3-(2-methoxyethyl)-2-oxo-2,3-dihydro-1,3-benzoxazol-5-yl]phenyl}ethyl]-1,4-oxazepane-2-carboxamide,

(2S)-N-{(1S)-1-Cyano-2-[4-(5-cyanothiophen-2-yl)phenyl]ethyl}-1,4-oxazepane-2-carboxamide,

(2S)-N-[(1S)-2-(4′-Carbamoyl-3′-fluorobiphenyl-4-yl)-1-cyanoethyl]-1,4-oxazepane-2-carboxamide,

(2S)-N-{(1S)-1-Cyano-2-[4-(1-methyl-2-oxo-1,2-dihydroquinolin-7-yl)phenyl]ethyl}-1,4-oxazepane-2-carboxamide,

(2S)-N-[(1S)-1-Cyano-2-{4-[2-oxo-3-(tetrahydro-2H-pyran-4-ylmethyl)-2,3-dihydro-1,3-benzoxazol-5-yl]phenyl}ethyl]-1,4-oxazepane-2-carboxamide,

(2S)-N-{(1S)-2-[4-(7-Chloro-3-methyl-2-oxo-2,3-dihydro-1,3-benzoxazol-5-yl)phenyl]-1-cyanoethyl}-1,4-oxazepane-2-carboxamide,

(2S)-N-[(1S)-1-Cyano-2-{4-[3-(2,2-difluoroethyl)-2-oxo-2,3-dihydro-1,3-benzoxazol-5-yl]phenyl}ethyl]-1,4-oxazepane-2-carboxamide,

(2S)-N-[(1S)-1-Cyano-2-{4-[2-oxo-3-(2,2,2-trifluoroethyl)-2,3-dihydro-1,3-benzoxazol-5-yl]phenyl}ethyl]-1,4-oxazepane-2-carboxamide,

(2S)-N-{(1S)-1-Cyano-2-[4-(3-methyl-2-oxo-2,3-dihydro-1,3-benzothiazol-5-yl)phenyl]ethyl}-1,4-oxazepane-2-carboxamide,

(2S)-N-{(1S)-1-Cyano-2-[4′-(methylsulfonyl)biphenyl-4-yl]ethyl}-1,4-oxazepane-2-carboxamide,

(2S)-N-{(1S)-2-[4′-(Azetidin-1-ylsulfonyl)biphenyl-4-yl]-1-cyanoethyl}-1,4-oxazepane-2-carboxamide,

(2S)-N-[(1S)-1-Cyano-2-(4′-fluorobiphenyl-4-yl)ethyl]-1,4-oxazepane-2-carboxamide,

(2S)-N-{(1S)-2-[4-(1,3-Benzothiazol-5-yl)phenyl]-1-cyanoethyl}-1,4-oxazepane-2-carboxamide, or

(2S)-N-[(1S)-1-Cyano-2-(4′-cyanobiphenyl-4-yl)ethyl]-1,4-oxazepane-2-carboxamide,

or a pharmaceutically acceptable salt of one of the foregoing compounds.

The treatment methods provided herein comprise the administration of a composition comprising an effective amount of a compound of formula (I), or a pharmaceutically acceptable salt thereof, to a patient in need of treatment of an ANCA associated vasculitis. The compounds of formula (I) and their pharmaceutically acceptable salts are inhibitors of dipeptidyl peptidase 1 (DPP1) activity. In one embodiment, the compound is INS1007, or a pharmaceutically acceptable salt thereof.

Administration routes include oral administration. Administration schedules can be determined by the user of the method, e.g., a prescribing physician. In one embodiment, administration is once daily. In another embodiment, administration is twice daily. In another embodiment, administration 1× daily, once every other day, once every third day, once every fourth day, 2× weekly, 3× weekly or 4× weekly.

In one embodiment, a composition comprising an effective amount of a compound of Formula (I) is administered orally. In a further embodiment, the compound of Formula (I) is INS1007, or a pharmaceutically acceptable salt thereof. In yet a further embodiment, administration is 1× daily, twice daily, once every other day, once every third day, once every fourth day, 2× weekly, 3× weekly or 4× weekly. In even a further embodiment, administration is once daily. In even a further embodiment, administration is oral once daily.

The term “treating” in one embodiment, includes: (1) preventing or delaying the appearance of clinical symptoms of the state, disorder or condition developing in the patient that may be afflicted with or predisposed to the state, disorder or condition but does not yet experience or display clinical or subclinical symptoms of the state, disorder or condition; (2) inhibiting the state, disorder or condition (e.g., arresting, reducing or delaying the development of the disease, or a relapse thereof in case of maintenance treatment, of at least one clinical or subclinical symptom thereof); (3) relieving the condition (for example, by causing regression of the state, disorder or condition or at least one of its clinical or subclinical symptoms). In one embodiment, “treating” refers to inhibiting the state, disorder or condition (e.g., arresting, reducing or delaying the development of the disease, or a relapse thereof in case of maintenance treatment, of at least one clinical or subclinical symptom thereof). In a further embodiment, treating of a patient comprises maintaining remission of the ANCA associated vasculitis, e.g., GPA or MPA. In a further embodiment, the ANCA associated vasculitis is GPA. In another embodiment, “treating” refers to relieving the condition (for example, by causing regression of the state, disorder or condition or at least one of its clinical or subclinical symptoms).

In one embodiment of the method of treatments provided herein, treating a patient for an ANCA associated vasculitis comprises decreasing the Birmingham Vasculitis Activity Score (BVAS) for the patient, as compared to the BVAS score prior to treatment (Suppiah et al. (2011). Rheumatology 50, pp. 899-905; Mukhtyar et al (2009). “Modification and validation of the Birmingham Vasculitis Activity Score” (version 3) ARD 2009 68:1827, each incorporated by reference herein in its entirety for all purposes). Such a decrease in one embodiment, can be a decrease to 0, i.e., where the treating achieves remission of the ANCA associated vasculitis.

In another embodiment, treating a patient comprises maintaining the BVAS score at 0, i.e., maintaining remission. The BVAS score measures the disease activity in patients with a variety of systemic vasculitides and scores abnormality ascribable to the presence of active vasculitis. Selga et al. (2006). Rheumatology 45, pp. 1276-1281, incorporated by reference herein in its entirety for all purposes.

The BVAS evaluation form includes: 56 disease items characterized in 9 groups and an “other” section. Items on the BVAS evaluation form are counted only if they are attributable to active vasculitis. The maximum possible score is 63. A score of 0 indicates disease remission, while a score of >1 indicates an active disease state (Suppiah et al. (2011). Rheumatology 50, pp. 899-905, incorporated by reference herein in its entirety for all purposes).

In one embodiment, the ANCA associated vasculitis is microscopic polyangiitis (MPA) and a method is provided for treating a patient in MPA remission and maintaining the remission in the patient. The method comprises administering to the patient a composition comprising an effective amount of a compound of Formula (I), or a pharmaceutically acceptable salt thereof. Remission, in one embodiment is measured by a BVAS score of 0 prior to the treatment and a BVAS score of 0 during the treatment, or subsequent to the treatment. In a further embodiment, the compound of Formula (I) is INS1007, or a pharmaceutically acceptable salt thereof. In one embodiment, the composition is administered orally. In yet a further embodiment, administration is 1× daily, twice daily, once every other day, once every third day, once every fourth day, 2× weekly, 3× weekly or 4× weekly. In even a further embodiment, administration is 1× daily. In one embodiment, administration is oral, 1× daily.

In one embodiment, the ANCA associated vasculitis is microscopic polyangiitis (MPA), and a method for treating an MPA patient comprises decreasing the patient's BVAS score to 0, from a BVAS score of >1. The method comprises administering to the patient a composition comprising an effective amount of a compound of Formula (I). In a further embodiment, the compound of Formula (I) is INS1007, or a pharmaceutically acceptable salt thereof. In one embodiment, the composition is administered orally. In yet a further embodiment, administration is 1× daily, twice daily, once every other day, once every third day, once every fourth day, 2× weekly, 3× weekly or 4× weekly. In even a further embodiment, administration is 1× daily. In even a further embodiment, administration is oral, 1× daily.

In one embodiment, the ANCA associated vasculitis is granulomatosis with polyangiitis (GPA), and a method for treating GPA in a patient in need thereof is provided. The patient may be in an active disease state or in remission, for example, as measured by the Birmingham Vasculitis Activity Score specific for Wegener's granulomatosis (BVAS/WG). Stone et al. (2001). A disease-specific activity index for Wegener's Granulomatosis. Arthritis & Rheumatism 44(4), pp. 912-920, incorporated by reference herein in its entirety for all purposes.

The BVAS/WG evaluation form includes: (1) 34 separate disease items, categorized into 9 groups; (2) an “other” section; (3) an asterisk by the 15 major items (i.e., an item that constitutes an immediate threat to the patient's life or to the function of a vital organ); (4) tick boxes to indicate new/worse or persistent disease; (5) an area to total the scores; (6) a section for the designation of disease status; (7) the physician's global assessment (PGA) of disease activity scale; and (8) a box for administrative use that contains information about the patient identification code and clinical center. Items on the BVAS/WG evaluation form are counted only if they result from active WG, and not from damage from previously active WG or another medical condition. The BVAS/WG includes categorical ratings that incorporate major and minor items into the definitions of disease status. Stone et al. (2001). Arthritis & Rheumatism 44(4), pp. 912-920, incorporated by reference herein in its entirety for all purposes. The four disease statuses are as follows:
(1) severe disease/flare (occurrence of any new/worse item that is major),
(2) limited disease/flare (occurrence of any new/worse item that is minor),
(3) persistent disease (presence of ≥1 item representing active disease that has continued since the patient's previous evaluation), and
(4) remission (no active disease; that is, no new/worse and no persistent items present).

The BVAS/WG score is calculated by multiplying the number of major items (either new/worse or persistent) by 3 and adding this number to the total number of minor items. The maximum BVAS/WG score, therefore, is 68, assuming that not more than 1 major and 1 minor “other” items are present. Stone et al. (2001). Arthritis & Rheumatism 44(4), pp. 912-920, incorporated by reference herein in its entirety for all purposes.

For a patient in an active disease state, in one embodiment, the method comprises decreasing the Birmingham Vasculitis Activity Score specific for Wegener's granulomatosis (BVAS/WG) for the patient, as compared to the BVAS/WG score prior to the treatment. In a further embodiment, a composition comprising an effective amount of a compound of Formula (I) is administered orally. In a further embodiment, the compound of Formula (I) is INS1007, or a pharmaceutically acceptable salt thereof. In yet a further embodiment, administration is 1× daily, twice daily, once every other day, once every third day, once every fourth day, 2× weekly, 3× weekly or 4× weekly. In yet a further embodiment, administration is 1× daily. In even a further embodiment, administration is oral, once daily.

In one embodiment of the method of treatments provided herein, treating GPA in a patient in need thereof comprises inhibiting a GPA flare. A GPA flare, as used herein, in one embodiment, is defined as an increase in the BVAS/WG score of 1 point or more. In a further embodiment, a composition comprising an effective amount of a compound of Formula (I) is administered orally to inhibit the GPA flare. In a further embodiment, the compound of Formula (I) is INS 1007, or a pharmaceutically acceptable salt thereof. In yet a further embodiment, administration is 1× daily, twice daily, once every other day, once every third day, once every fourth day, 2× weekly, 3× weekly or 4× weekly. In yet a further embodiment, administration is 1× daily. In even a further embodiment, administration is oral once daily.

In another embodiment, the ANCA associated vasculitis is granulomatosis with polyangiitis (GPA), and the patient is in GPA remission, as indicated by, for example, a BVAS/WG of 0. The method in one embodiment comprises maintaining remission of the GPA in the patient. The patient may be in remission for at least 30, 60, 90, or 120 days prior to treatment. The patient, in one embodiment, maintains remissions during treatment and/or subsequent to treatment. Remission can be measured, in one embodiment, 1 day, 7 days, 14 days, 30 days, 60 days, 90 days or 120 days subsequent to the treatment. The patient treated with one of the methods provided herein, in one embodiment, has undergone or is currently undergoing GPA therapy with an anti-CD20 antibody (e.g., rituximab), cyclophosphamide, or a steroid (e.g., a corticosteroid, such as a glucocorticoid). In one embodiment, the compound of Formula (I) is INS 1007, or a pharmaceutically acceptable salt thereof. In one embodiment, the compostion is administered orally. In yet a further embodiment, administration is 1× daily, twice daily, once every other day, once every third day, once every fourth day, 2× weekly, 3× weekly or 4× weekly. In yet a further embodiment, administration is 1× daily. In even a further embodiment, administration is oral once daily. In one embodiment, the INS1007 or a pharmaceutically acceptable salt thereof is administered orally at from about 10 mg to about 50 mg, or from about 20 mg to about 45 mg, e.g., 10 mg, 25 mg, 30 mg, or 40 mg, once daily. In another embodiment, the INS1007 is administered orally at 40 mg once daily.

The treatment methods of the present disclosure can be employed to treat a patient in GPA remission to maintain the remission. Alternatively, the methods of the present disclosure can be employed to treat a patient with active GPA, in order to affect remission in the patient.

In some embodiments, treatment efficacy is defined by a lack of relapse, or a slowing of a relapse, as compared to a patient not treated via one of the methods provided herein. The relapse may be a major relapse or a minor replapse. A major relapse is defined as reappearance or worsening of disease with (1) a BVAS/WG>0 and involvement of at least one major organ, (2) a life-threatening manifestation, or (3) both (1) and (2). A minor relapse is defined as reappearance or worsening of disease with a BVAS/WG>0, not corresponding to a major relapse, but requiring mild treatment intensification.

Other measures of efficacy include rate of flares, time to relapse, change in Vasculitis Damage Index (VDI) from baseline, systemic corticosteroid use based on total oral corticosteroid dose and duration of oral corticosteroid use, and change in quality of life from baseline measured by, e.g., the short form health survey questionnaire (SF-36) score (discussed below).

VDI is a standardized clinical measure of damage in the systemic vasculitides. See Exley et al. (1997), Arthritis Rheum. 40(2):371-80, incorporated herein by reference in its entirety. VDI records the presence or absence of 64 items of damage separated into eleven groupings, which include ten organ-based systems and one general category as follows:

(1) Musculoskeletal; (2) Skin; (3) Ear, nose, and throat; (4) Pulmonary; (5) Cardiovascular; (6) Renal; (7) Gastrointestinal; (8) Peripheral vascular; (9) Ocular; (10) Neuropsychiatric; and (11) Other damage.

The VDI score is the simple sum of the damage items and cumulative, i.e., all previously scored items are carried over to each subsequent assessment, and thus can only remain stable or increase.

In another embodiment of the method of treatments provided herein, treating a patient in need thereof comprises improving the short form health survey questionnaire (SF-36) score for the patient, as compared to the SF-36 score of the patient prior to treatment. The SF-36 measures eight scales: physical functioning (PF), role physical (RP), bodily pain (BP), general health (GH), vitality (VT), social functioning (SF), role emotional (RE), and mental health (MH). See, e.g., Lins and Carvalho (2016). SAGE Open Medicine 4, pp. 1-12, incorporated by reference herein in its entirety for all purposes. In a further embodiment, a composition comprising an effective amount of a compound of Formula (I) is administered orally. In a further embodiment, the compound of Formula (I) is INS1007, or a pharmaceutically acceptable salt thereof. In yet a further embodiment, administration is 1× daily, twice daily, once every other day, once every third day, once every fourth day, 2× weekly, 3× weekly or 4× weekly. In yet a further embodiment, administration is 1× daily. In even a further embodiment, administration is oral once daily.

In another embodiment of the method for treating an ANCA associated vasculitis provided herein, a composition comprising an effective amount of a compound of formula (I), or a pharmaceutically acceptable salt thereof, is administered to a patient in need thereof. The method comprises decreasing intra-leukocyte proteinase 3 (PR3) activity, as compared to the patient's intra-leukocyte PR3 activity, prior to treatment. The compound of formula (I) in a further embodiment, is INS1007, or a pharmaceutically acceptable salt thereof. In one embodiment, the composition is administered orally to the patient in need of treatment. In yet a further embodiment, administration is 1× daily, twice daily, once every other day, once every third day, once every fourth day, 2× weekly, 3× weekly or 4× weekly. In yet a further embodiment, administration is 1× daily. In even a further embodiment, administration is oral once daily.

In one embodiment, the PR3 activity is measured in leukocytes (e.g., neutrophils) obtained from the patient's whole blood. In another embodiment, the PR3 activity is measured in leukocytes (e.g., neutrophils) obtained from the patient's sputum. In one embodiment, the decreasing is by about 10%, about 20%, about 25%, about 30%, about 40%, about 50%, about 60%, about 70%, or about 80%. In another embodiment, decreasing PR3 activity comprises decreasing by at least about 1%, at least about 5%, at least about 10%, at least about 20%, at least about 30%, at least about 40%, at least about 50%, at least about 60%, at least about 70%, or at least about 80%. In one embodiment, the compound of formula (I) is INS1007, or a pharmaceutically acceptable salt thereof. In one embodiment, the composition is administered orally. In yet a further embodiment, administration is 1× daily, twice daily, once every other day, once every third day, once every fourth day, 2× weekly, 3× weekly or 4× weekly. In yet a further embodiment, administration is 1× daily. In even a further embodiment, administration is oral once daily.

In another embodiment of the method of treatments provided herein, treating a patient in need thereof comprises administering to the patient a pharmaceutical composition comprising an effective amount of a compound of Formula (I), or a pharmaceutically acceptable salt thereof, and decreasing the neutrophil cell surface expression of proteinase 3 of the patient, as compared to the neutrophil cell surface expression of proteinase 3 prior to treatment. In one embodiment, decreasing comprises decreasing the PR3 neutrophil cell surface expression by about 10%, about 20%, about 25%, about 30%, about 40%, about 50%, about 60%, about 70%, or about 80%. In another embodiment, decreasing proteinase 3 cell surface expression comprises decreasing by at least about 1%, at least about 5%, at least about 10%, at least about 20%, at least about 30%, at least about 40%, at least about 50%, at least about 60%, at least about 70% or at least about 80%. In one embodiment, the compound of formula (I) is INS 1007, or a pharmaceutically acceptable salt thereof. In a further embodiment, the composition is administered orally. In yet a further embodiment, administration is 1× daily, twice daily, once every other day, once every third day, once every fourth day, 2× weekly, 3× weekly or 4× weekly. In yet a further embodiment, administration is 1× daily. As such, in one embodiment, the composition is administered orally and once daily.

In another embodiment of the method for treating an ANCA associated vasculitis (e.g., GPA or MPA), a composition comprising an effective amount of a compound of formula (I), or a pharmaceutically acceptable salt thereof, is administered to a patient in need of the treatment, wherein the method comprises decreasing the neutrophil serine protease (NSP) activity in the patient's blood, as compared to the patient's NSP activity, prior to treatment. The compound of formula (I) in one embodiment is INS1007, or a pharmaceutically acceptable salt thereof. In one embodiment, the composition is administered orally. In one embodiment, administration 1× daily, twice daily, once every other day, once every third day, once every fourth day, 2× weekly, 3× weekly or 4× weekly. In a further embodiment, administration is 1× daily. In a further embodiment, administration is oral once daily. In even a further embodiment, the compound of formula (I) is INS1007. The NSP may be neutrophil elastase (NE), proteinase 3 (PR3) and/or cathepsin G (CatG). In one embodiment, decreasing NSP activity is by about 10%, about 20%, about 25%, about 30%, about 40%, about 50%, about 60%, about 70%, or about 80%. In another embodiment, decreasing NSP activity comprises decreasing NSP activity by at least about 1%, at least about 5%, at least about 10%, at least about 20%, at least about 30%, at least about 40%, at least about 50%, at least about 60%, at least about 70%, or at least about 80%.

In yet another embodiment of the method for treating an ANCA associated vasculitis (e.g., GPA or MPA) provided herein, a composition comprising an effective amount of a compound of formula (I), or a pharmaceutically acceptable salt thereof, is administered to a patient in need thereof, wherein the method comprises decreasing the patient's ANCA blood concentration, as compared to the patient's ANCA blood concentration prior to treatment. In one embodiment, the composition is administered via oral administration. The compound of formula (I) in one embodiment is INS1007, or a pharmaceutically acceptable salt thereof. In one embodiment, the ANCA blood concentration is measured in the patient's blood plasma or blood serum. In a further embodiment, administration is 1× daily, twice daily, once every other day, once every third day, once every fourth day, 2× weekly, 3× weekly or 4× weekly. In one embodiment, administration is oral once daily.

In one embodiment, the method comprises decreasing the ANCA blood concentratoin of the patient by about 10%, about 20%, about 25%, about 30%, about 40%, about 50%, about 60%, about 70%, or about 80%. In another embodiment, decreasing ANCA blood concentration comprises decreasing by at least about 1%, at least about 5%, at least about 10%, at least about 20%, at least about 30%, at least about 40%, at least about 50%, at least about 60%, at least about 70%, or at least about 80%. In one embodiment, the ANCA blood concentration is measured in the patient's blood plasma or blood serum.

In one embodiment, decreasing the ANCA antibody concentration comprises decreasing the PR3 ANCA antibody concentration in the patient, as compared to the PR3 ANCA antibody concentration prior to treatment. In another embodiment, decreasing the ANCA antibody concentration comprises decreasing the MPO ANCA antibody concentration in the patient, as compared to the MPO ANCA antibody concentration prior to treatment.

In yet another embodiment of the method for treating an ANCA associated vasculitis (e.g., GPA or MPA) provided herein, a composition comprising an effective amount of a compound of formula (I), or a pharmaceutically acceptable salt thereof, is administered to a patient in need thereof, wherein the method comprises decreasing the number of CD19+ B-cells in the patient, as compared to the number of CD19+ B-cells in the patient, prior to treatment. The compound of formula (I), in one embodiment, is INS 1007, or a pharmaceutically acceptable salt thereof. In one embodiment, the composition is administered via oral administration. In a further embodiment, administration is 1× daily, twice daily, once every other day, once every third day, once every fourth day, 2× weekly, 3× weekly or 4× weekly. In one embodiment, administration 1× daily. In another embodiment, administration is oral once daily.

The dosage administered will vary with the compound employed, the mode of administration, the treatment desired and the disorder indicated. In one embodiment, if the compound is administered orally, then the daily dosage of the compound of the disclosure may be in the range from 0.01 micrograms per kilogram body weight (m/kg) to 100 milligrams per kilogram body weight (mg/kg). In one embodiment, the compound is INS1007, and the daily dosage of INS1007 is from about 10 mg to 50 mg, or from about 20 mg to about 45 mg. For example, the daily dose in one embodiment is 10 mg, 25 mg, 30 mg or 40 mg. In a further embodiment, INS1007 is administered orally. In a further embodiment, the compound is INS 1007, and the daily dosage of INS1007 is 40 mg administered orally.

In one embodiment, the compound of Formula (I) is administered in an oral dosage form. In a further embodiment, the compound of Formula (I) is administered as a 10 mg to 50 mg dosage form, for example, a 5 mg dosage form, a 10 mg dosage form, a 15 mg dosage form, a 20 mg dosage form, a 25 mg dosage form, a 30 mg dosage form, a 35 mg dosage form, a 40 mg dosage form, a 45 mg dosage form or a 50 mg dosage form. In a further embodiment, the dosage form is a 10 mg, 25 mg or 40 mg dosage form. In a further embodiment, the dosage form is administered once daily. In even a further embodiment, the compound is (2S)-N-{(1S)-1-cyano-2-[4-(3-methyl-2-oxo-2,3-dihydro-1,3-benzoxazol-5-yl)phenyl]ethyl}-1,4-oxazepane-2-carboxamide (INS 1007), or a pharmaceutically acceptable salt thereof.

The compounds of formula (I), or pharmaceutically acceptable salts thereof, may be used on their own but will generally be administered in the form of a pharmaceutical composition in which the formula (I) compound/salt (active ingredient) is in a composition comprising a pharmaceutically acceptable adjuvant(s), diluents(s) and/or carrier(s). Conventional procedures for the selection and preparation of suitable pharmaceutical formulations are described in, for example, “Pharmaceuticals—The Science of Dosage Form Designs”, M. E. Aulton, Churchill Livingstone, 2^{nd} Ed. 2002, incorporated by reference herein in its entirety for all purposes.

Depending on the mode of administration, the pharmaceutical composition will comprise from 0.05 to 99%w (percent by weight), for example, from 0.05 to 80%w, or from 0.10 to 70%w, or from 0.10 to 50%w, of active ingredient, all percentages by weight being based on total composition.

In one oral administration embodiment, the oral dosage form is a film-coated oral tablet. In a further embodiment, the dosage form is an immediate release dosage form with rapid dissolution characteristics under in vitro test conditions.

In one embodiment, the oral dosage form is administered once daily. In a further embodiment, the oral dosage form is administered at approximately the same time every day, e.g., prior to breakfast. In another embodiment, the composition comprising an effective amount of formula (I) is administered 2× day. In yet another embodiment, the composition comprising an effective amount of a compound of formula (I) or a pharmaceutically acceptable salt thereof is administered once-a-week, every other day, every third day, 2× week, 3× week, 4× week, or 5× week.

For oral administration the compound of the disclosure may be admixed with adjuvant(s), diluent(s) or carrier(s), for example, lactose, saccharose, sorbitol, mannitol; starch, for example, potato starch, corn starch or amylopectin; cellulose derivative; binder, for example, gelatine or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatine, talcum and titanium dioxide.

For the preparation of soft gelatine capsules, the compound of the disclosure may be admixed with, for example, a vegetable oil or polyethylene glycol. Hard gelatine capsules may contain granules of the compound using pharmaceutical excipients like the above-mentioned excipients for tablets. Also liquid or semisolid formulations of the compound of the disclosure may be filled into hard gelatine capsules.

In one embodiment, the composition is an oral disintegrating tablet (ODT). ODTs differ from traditional tablets in that they are designed to be dissolved on the tongue rather than swallowed whole

In one embodiment, the composition is an oral thin film or an oral disintegrating film (ODF). Such formulations, when placed on the tongue, hydrate via interaction with saliva, and release the active compound from the dosage form. The ODF, in one embodiment, contains a film-forming polymer such as hydroxypropylmethylcellulose (HPMC), hydroxypropyl cellulose (HPC), pullulan, carboxymethyl cellulose (CMC), pectin, starch, polyvinyl acetate (PVA) or sodium alginate.

Liquid preparations for oral application may be in the form of syrups, solutions or suspensions. Solutions, for example, may contain the compound of the disclosure, the balance being sugar and a mixture of ethanol, water, glycerol and propylene glycol. Optionally such liquid preparations may contain coloring agents, flavoring agents, saccharine and/or carboxymethylcellulose as a thickening agent. Furthermore, other excipients known to those skilled in art may be used when making formulations for oral use.

A compound of formula (I), or a pharmaceutically acceptable salt thereof, may also be administered in conjunction with a further compound used for the treatment of an ANCA associated vasculitis (e.g., GPA or MPA) via one of the methods described herein.

The further compound is administered concurrently, sequentially or in admixture with a compound of Formula (I), for the treatment of an ANCA associated vasculitis.

The further compound, in one embodiment, is an anti-TNF-α antibody, e.g., infliximab, adalimumab, certolizumab pegol, and golimumab. In a further embodiment, the anti-TNF-α antibody is infliximab.

The further compound, in another embodiment, is an anti-CD20 antibody, e.g., rituximab, ocrelizumab, obinutuzumab, ofatumumab, ibritumomab tiuxetan, tositumomab, and ublituximab. In a further embodiment, the anti-CD20 antibody is rituximab.

In yet another embodiment, the further compound is a steroid. In a further embodiment, the steroid is a corticosteroid. In even a further embodiment, the further compound is a glucocorticoid.

In even another embodiment, the further compound is cyclophosphamide (CYC), alone or in combination with one or more glucocorticoids.

In one combination therapy embodiment, the compound of the disclosure, or a pharmaceutically acceptable salt thereof, is administered concurrently or sequentially with one or more further active ingredients selected from one or more of those provided above. For example, the compound of Formula (I), or a pharmaceutically acceptable salt thereof, may be administered concurrently or sequentially with a further pharmaceutical composition for use as a medicament for the treatment of an ANCA associated vasculitis. The further pharmaceutical composition may be a medicament which the patient may already be prescribed (e.g., an existing standard of care medication), and may itself be a composition comprising one or more active ingredients selected from those defined above.

Formula I, its subgenera, and INS1007, as well as methods of making the same, are disclosed in U.S. Pat. No. 9,522,894, the disclosure of which is incorporated by reference in its entirety for all purposes.

A compound of formula (I) or a pharmaceutically acceptable salt thereof can be synthesized by reacting a compound of formula (II),

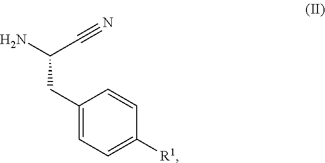

wherein R¹ is as defined in formula (I), with a compound of formula (III),

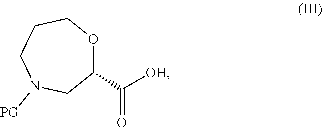

wherein PG represents a protecting group (e.g. tert-butoxycarbonyl), and optionally thereafter carrying out one or more of the following procedures:
converting a compound of formula (I) into another compound of formula (I);
removing any protecting groups; and/or
forming a pharmaceutically acceptable salt.

The process is conveniently carried out in the presence of a base such as DiPEA or TEA and one or more activating agents such as EDCI, 2-pyridinol-1-oxide, or T3P. The reaction is conveniently carried out in an organic solvent such as DMF or DCM at a temperature, for example, in the range from about 20° C. to about 100° C., from about 20° C. to about 80° C., from about 20° C. to about 60° C., or from about 20° C. to about 40° C., in particular at ambient temperature (about 25° C.).

Compounds of formula (II) may be prepared by reaction of a compound of formula (IV),

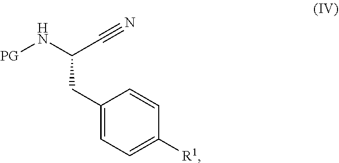

wherein PG represents a protecting group (e.g. tert-butoxycarbonyl), with a suitable reagent to remove the protecting group PG. An example of a suitable reagent is formic acid.

Compounds of formula (IV) may be prepared by reacting a compound of formula (V),

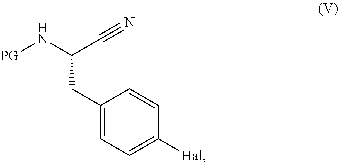

wherein PG represents a protecting group (e.g., tert-butoxycarbonyl) and Hal represents a halogen (e.g. I or Br), with a compound of formula (VI) or an ester thereof,

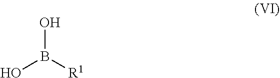

wherein R¹ is as defined in formula (I), in the presence of a catalyst such as Pd(dppf)Cl₂·DCM or 1,1 bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from about 20° C. to about 100° C., from about 40° C. to about 90° C., or from about 60° C. to about 80° C., particularly at about 75° C.

Compounds of formula (V) may be prepared from a compound of formula (VII),

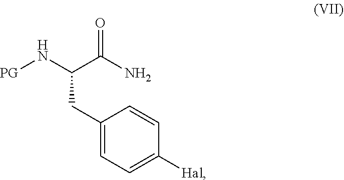

in which PG represents a protecting group (e.g. tert-butoxycarbonyl) and Hal represents a halogen (e.g., I or Br), using standard literature procedures for the dehydration of an amide, for example with Burgess reagent, or with a reagent such as T3P with or without a base such as DiPEA, in a solvent such as DCM or DMF at a temperature in the range of from about −20° C. to about 100° C., from about −20° C. to about 80° C., from about −20° C. to about 50° C., from about −20° C. to about 20° C., from about −10° C. to about 10° C., for example at about 0° C.

Compounds of formula (VII) may be prepared by reacting a compound of formula (VIII),

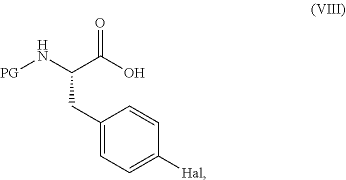

in which PG represents a protecting group (e.g. tert-butoxycarbonyl) and Hal represents a halogen (e.g., I or Br), with an aqueous ammonia solution, using standard literature procedures for the formation of an amide, for example, in the presence of a base such as N-ethyl-morpholine or DiPEA and an activating agent such as TBTU or T3P. The reaction is conveniently carried out in an organic solvent such as DMF, at a temperature in the range of from about −20° C. to about 100° C., from about −20° C. to about 80° C., from about −20° C. to about 50° C., from about −20° C. to about 20° C., from about −10° C. to about 10° C., for example at about 0° C.

Compounds of formula (VIII) are either commercially available, are known in the literature (e.g., from Tetrahedron:Asymmetry, 1998, 9, 503, incorporated by reference herein in its entirety for all purposes) or may be prepared using known techniques.

There is further provided a process for the preparation of a compound of formula (I), or a pharmaceutically acceptable salt thereof, as defined above which comprises reacting a compound of formula (IX),

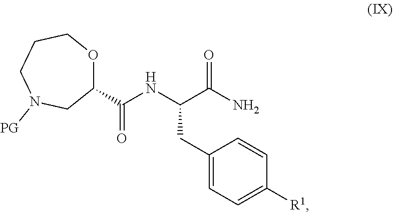

wherein R¹ is as defined above and PG represents a protecting group (e.g. tert-butoxycarbonyl), using standard literature procedures for the dehydration of an amide, for example with Burgess reagent or with a reagent such as T3P with or without a base such as DiPEA, in a solvent such as DCM or DMF at a temperature in the range from about −20° C. to about 100° C., from about 20° C. to about 80° C., from about 20° C. to about 60° C., from about 20° C. to about 40° C., for example at about 25° C., and thereafter reacting with a suitable reagent to remove the protecting group PG. An example of a suitable reagent is formic acid.

A compound of formula (IX) may be prepared by reacting a compound of formula (X), wherein PG represents a protecting group (e.g. tert-butoxycarbonyl),

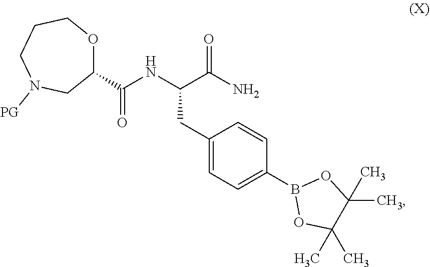

with a halide of formula (XI), wherein R¹ is defined as in formula (I), R¹—Br/I (XI), in the presence of a catalyst such as bis[bis(1,2-diphenylphosphino)ethane]palladium(0), or Pd(dppf)Cl₂ DCM, and a base such as potassium carbonate or sodium carbonate. The reaction is conveniently carried out in a solvent such as dioxane/water mixture or ACN/water mixture at a temperature, for example, in the range from about 20° C. to about 100° C., from about 40° C. to about 90° C., or from about 60° C. to about 90° C., particularly at about 80° C.

A compound of formula (X) may be prepared by reacting a compound of formula (XII), wherein PG represents a protecting group (e.g. tert-butoxycarbonyl),

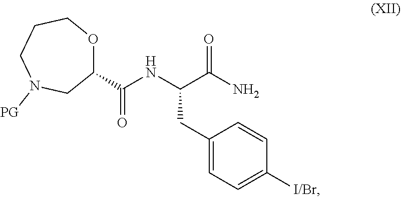

with B₂Pin₂ in the presence of a suitable catalyst such as Pd(dppf)Cl₂.DCM and with or without 1,1′-bis(diphenylphosphino)ferrocene or 1,1-bis(di-tert-butylphosphino)ferrocene palladium dichloride, with a suitable salt such as potassium acetate, in a solvent such as DMSO at a temperature in the range from about 60° C. to about 100° C., from about 70° C. to about 90° C., for example at about 85° C.

A compound of formula (XII) may be prepared by reacting a compound of formula (XIII),

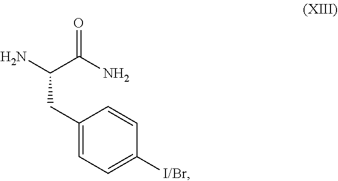

with a compound of formula (III),

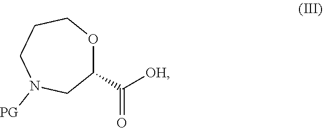

wherein PG represents a protecting group (e.g. tert-butoxycarbonyl) in the presence of a base such as DiPEA or TEA and an activating agent such as EDCI, 2-pyridinol-1-oxide, or T3P. The reaction is conveniently carried out in an organic solvent such as DMF or DCM at a temperature, for example, in the range from about 20° C. to about 100° C., from about 20° C. to about 80° C., from about 20° C. to about 60° C., from about 20° C. to about 40° C., for example, at ambient temperature (about 25° C.).

Compounds of formula (XIII) may be prepared by reacting a compound of formula (XIV),

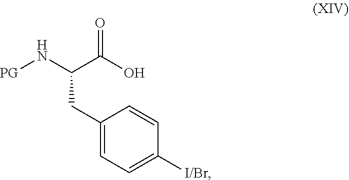

in which PG is as defined in formula (VII), with an aqueous ammonia solution, using standard literature procedures for the formation of an amide, for example, in the presence of a base such as N-ethyl-morpholine or DiPEA and an activating agent such as a “uronium” reagent (for example TBTU), or T3P. The reaction is conveniently carried out in an organic solvent such as DMF, at a temperature in the range from about −20° C. to about 100° C., from about −20° C. to about 80° C., from about −20° C. to about 50° C., from about −20° C. to about 20° C., from about −10° C. to about 10° C., for example at about 0° C.

A compound of formula (IX) may be prepared by reacting a compound of formula (XII) wherein PG represents a protecting group (e.g. tert-butoxycarbonyl), with a compound of formula (VI) or a boronate ester thereof, in the presence of a catalyst such as bis[bis(1,2-diphenylphosphino)ethane]palladium(0) or Pd(dppf)Cl₂.DCM and a base such as potassium carbonate or sodium carbonate. The reaction is conveniently carried out in a solvent such as dioxane/water or ACN/water mixture at a temperature, for example, in the range from about 20° C. to about 100° C., from about 40° C. to about 90° C., or from about 60° C. to about 90° C., for example, at about 80° C.

There is further provided a process for the preparation of a compound of formula (I), or a pharmaceutically acceptable salt thereof, as defined above which comprises reacting a compound of formula (XV),

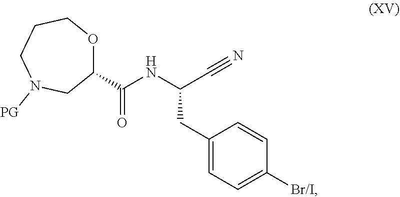

wherein PG represents a protecting group (e.g. tert-butoxycarbonyl), with a compound of formula (VI) or an ester thereof, wherein R¹ is as defined in formula (I), in the presence of a catalyst such as Pd(dppf)Cl₂.DCM or 1,1 bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from about 20° C. to about 100° C., from about 40° C. to about 90° C., or from about 60° C. to about 80° C., particularly at about 75° C., and thereafter reacting with a suitable reagent to remove the protecting group PG. An example of a suitable reagent is formic acid.

Compounds of formula (XV) may be prepared from compounds of formula (XII) using standard procedures for the dehydration of an amide, for example with Burgess reagent or a reagent such as TBTU or T3P with or without a base such as DiPEA, in a solvent such as DCM or DMF at a temperature in the range from about −20° C. to about 100° C., from about 20° C. to about 80° C., from about 20° C. to about 60° C., from about 20° C. to about 40° C., for example at about 25° C.

There is further provided a process for the preparation of a compound of formula (I) or a pharmaceutically acceptable salt thereof as defined above which comprises reacting a compound of formula (XVI),

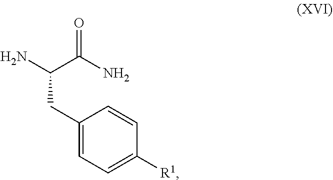

wherein R¹ is as defined in formula (I), with a compound of formula (III), conveniently carried out in the presence of a base such as DiPEA or TEA and one or more activating agents such as EDCI, 2-pyridinol-1-oxide, or T3P, followed by a dehydrating reagent such as T3P. The reaction is conveniently carried out in an organic solvent such as DMF or DCM at a temperature, for example, in the range from about 20° C. to about 100° C., from about 20° C. to about 80° C., from about 20° C. to about 60° C., from about 20° C. to about 40° C., in particular at ambient temperature (about 25° C.).

Compounds of formula (XVI) can be prepared from reacting compounds of formula (VII) with compounds of formula (VI) or an ester thereof, wherein R¹ is as defined in formula (I), in the presence of a catalyst such as Pd(dppf)Cl₂.DCM or 1,1bis(di-tert-butylphosphino)ferrocene palladium dichloride and a base such as potassium carbonate or sodium carbonate. The reaction is conveniently carried out in a solvent such as dioxane/water mixture or ACN/water mixture at a temperature, for example, in the range from about 20° C. to about 100° C., from about 40° C. to about 90° C., or from about 60° C. to about 80° C., particularly at about 75° C., followed by deprotection of PG.

A compound of formula (III),

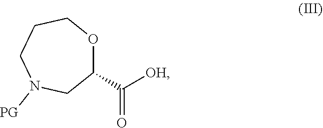

wherein PG represents a protecting group (e.g. tert-butoxycarbonyl) is either commercially available, or may be prepared from a compound of formula (XVII),

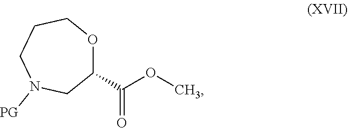

using literature procedures for mild ester hydrolysis (e.g. from Tetr. Lett., 2007, 48, 2497, incorporated by reference herein in its entirety for all purposes), for example with LiBr and a base such as TEA, in a solvent such as ACN/water mixture, for example at from about 0° C. to about 40° C., from about 5° C. to about 35° C., from about 10° C. to about 30° C., for example, at about 25° C.

A compound of formula (XVII), wherein PG represents a protecting group (e.g. tert-butoxycarbonyl), may be prepared from a compound of formula (XVIII),

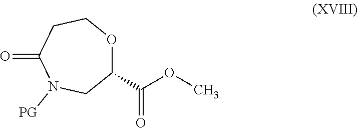

using a reducing agent, for example BH₃-DMS, in a solvent such as THF, at a temperature in the range of from about 0° C. to 40° C., from about 5° C. to about 35° C., from about 10° C. to 30° C., or at about 25° C.

A compound of formula (XVIII), where PG represents a protecting group (e.g. tert-butoxycarbonyl), may be prepared from a compound of formula (XIX), using a biocatalytic transformation for chemoselective lactam formation, e.g., using a lipase such as Novozym 435, in a solvent such as an ether, e.g., dioxane, at a temperature in the range from about 0° C. to 80° C., from about 20° C. to about 60° C., from about 40° C. to about 60° C., for example at about 55° C., followed by conditions for introduction of the protecting group PG.

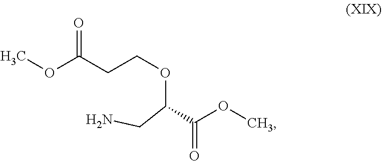

A compound of formula (XIX) may be prepared from a compound of formula (XX),

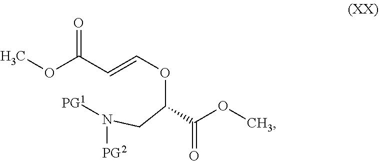

wherein PG¹ and PG² are protecting groups (e.g., benzyl), using conditions for hydrogenation, for example using H₂ (g), and a reagent such as palladium dihydroxide on carbon, in a solvent such as methanol or dioxane, under a pressure of, for example, 5-15 bar, 8-12 bar, for example, 10 bar, at a temperature in the range from about 25° C. to about 80° C., from about 25° C. to about 60° C., from about 30° C. to about 50° C., for example at about 40° C.

A compound of formula (XX), wherein PG¹ and PG² are protecting groups (e.g., benzyl), may be prepared from a compound of formula (XXI),

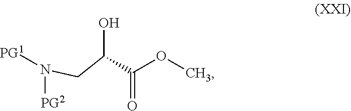

wherein PG¹ and PG² are protecting groups (e.g. benzyl), using conditions for Oxa-Michael reaction, reacting with methyl propynoate, in presence of a base such as 4-methylmorpholine, in a solvent such as toluene, at a temperature in the range from about 0° C. to about 100° C., from about 10° C. to about 80° C., from about 20° C. to about 60° C., from about 20° C. to about 40° C., for example at about 25° C.

A compound of formula (XXI), wherein PG¹ and PG² are protecting groups (e.g. benzyl), may be prepared from reacting a diprotected benzyl amine (e.g., dibenzylamine) with (S)-methyl oxirane-2-carboxylate, in a solvent such as ethanol, at a temperature in the range from about 0° C. to about 90° C., from about 0° C. to about 80° C., from about 20° C. to about 80° C., from about 40° C. to about 80° C., or from about 60° C. to about 78° C., for example, at 70° C.

Alternatively, a compound of formula (III),

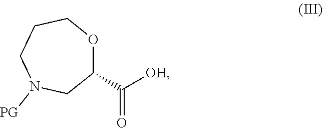

wherein PG represents a protecting group (e.g. tert-butoxycarbonyl) may be prepared from oxidation of a compound of formula (XXII),

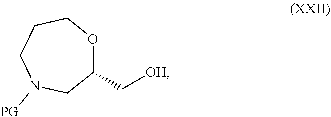

for example, using reagents such as TEMPO, and sodium hypochlorite, optionally in the prescence of a salt such as sodium bromide, in a solvent such as DCM/water, and in the presence of a buffer such as NaHCO₃, and a phase transfer catalyst such as tetrabutylammonium bisulphate, at a temperature in the range from about 0° C. to about 100° C., from about 10° C. to about 80° C., from about 20° C. to about 60° C., from about 20° C. to about 40° C., e.g., at 25° C.

A compound of formula (XXII), wherein PG represents a protecting group (e.g., tert-butoxycarbonyl) may be prepared from a compound of formula (XXIII),

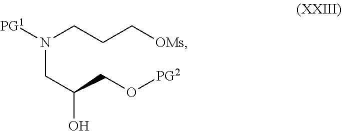

wherein PG¹ and PG² are protecting groups (e.g. benzyl), reacting with a base such as sodium hydride, in a solvent such as THF, at a temperature in the range from about 0° C. to about 60° C., from about 10° C. to about 50° C., from about 20° C. to about 40° C., from about 20° C. to about 30° C., e.g., at 25° C., followed by interconversion of protecting groups PG, PG¹ and PG², as defined in formula (XXII) and (XXIII).

A compound of formula (XXIII), wherein PG¹ and PG² are protecting groups (e.g., benzyl), may be prepared from reacting protected 3-aminopropanol (e.g. N-benzyl-3-aminopropanol) with (S)-2-((benzyloxy)methyl)oxirane, in a solvent such as ethanol or propanol, at a temperature in the range from about 0° C. to about 70° C., from about 10° C. to about 60° C., from about 30° C. to about 50° C., or at about 40° C., followed by reacting the crude product with methanesulfonyl chloride, in the prescence of a base such as DiPEA, in a solvent such as DCM, at a temperature in the range from about −10° C. to about 25° C., from about −10° C. to about 10° C., from about −10° C. to about 5° C., from about −8° C. to about 0° C., or e.g., at about −5° C.

Compounds of formula (VI) or an ester thereof, (VIII), (XI) and (XIV) are either commercially available, are known in the literature or may be prepared using known techniques.

It will be appreciated by those skilled in the art that in the processes of the present disclosure certain functional groups such as hydroxyl or amino groups in the reagents may need to be protected by protecting groups. Thus, the preparation of the compounds of formula (I) may involve, at an appropriate stage, the removal of one or more protecting groups.

The skilled person will recognise that at any stage of the preparation of the compounds of formula (I), mixtures of isomers (e.g., racemates) of compounds corresponding to any of formulae (II)-(V), (VII)-(X) and (XXII)-(XVI) may be utilized. At any stage of the preparation, a single stereoisomer may be obtained by isolating it from a mixture of isomers (e.g., a racemate) using, for example, chiral chromatographic separation.

The protection and deprotection of functional groups is described in ‘Protective Groups in Organic Synthesis’, 4^{th} Ed, T. W. Greene and P. G. M. Wuts, Wiley (2006) and ‘Protecting Groups’, 3^{rd} Ed P. J. Kocienski, Georg Thieme Verlag (2005), incorporated by reference herein in its entirety for all purposes.

As provided throughout, according to the methods provided herein, a compound of formula (I) can be administered as a pharmaceutically acceptable salt. A pharmaceutically acceptable salt of a compound of formula (I) may be advantageous due to one or more of its chemical or physical properties, such as stability in differing temperatures and humidities, or a desirable solubility in H₂O, oil, or other solvent. In some instances, a salt may be used to aid in the isolation or purification of the compound of formula (I).

Where the compound of formula (I) is sufficiently acidic, pharmaceutically acceptable salts include, but are not limited to, an alkali metal salt, e.g., Na or K, an alkali earth metal salt, e.g., Ca or Mg, or an organic amine salt. Where the compound of formula (I) is sufficiently basic, pharmaceutically acceptable salts include, but are not limited to, inorganic or organic acid addition salts.

There may be more than one cation or anion depending on the number of charged functions and the valency of the cations or anions.

For reviews on suitable salts, and pharmaceutically acceptable salts amenable for use herein, see Berge et al., J. Pharm. Sci., 1977, 66, 1-19 or “Handbook of Pharmaceutical Salts: Properties, selection and use”, P. H. Stahl, P.G. Vermuth, IUPAC, Wiley-VCH, 2002, incorporated by reference herein in its entirety for all purposes.

The compounds of formula (I) may form mixtures of its salt and co-crystal forms. It is also to be understood that the methods provided herein can employ such salt/co-crystal mixtures of the compound of formula (I).

Salts and co-crystals may be characterized using well known techniques, for example X-ray powder diffraction, single crystal X-ray diffraction (for example to evaluate proton position, bond lengths or bond angles), solid state NMR, (to evaluate for example, C, N or P chemical shifts) or spectroscopic techniques (to measure for example, O—H, N—H or COOH signals and IR peak shifts resulting from hydrogen bonding).

It is also to be understood that certain compounds of formula (I) may exist in solvated form, e.g., hydrates, including solvates of a pharmaceutically acceptable salt of a compound of formula (I).

In one embodiment, certain compounds of formula (I) may exist as racemates and racemic mixtures, single enantiomers, individual diastereomers and diastereomeric mixtures. It is to be understood that the present disclosure encompasses all such isomeric forms. Certain compounds of formula (I) may also contain linkages (e.g., carbon-carbon bonds, carbon-nitrogen bonds such as amide bonds) wherein bond rotation is restricted about that particular linkage, e.g. restriction resulting from the presence of a ring bond or double bond. Accordingly, it is to be understood that the methods provided herein can employ such isomers. Certain compound of formula (I) may also contain multiple tautomeric forms. It is to be understood that the present disclosure encompasses all such tautomeric forms. Stereoisomers may be separated using conventional techniques, e.g. chromatography or fractional crystallization, or the stereoisomers may be made by stereoselective synthesis.

In a further embodiment, the compounds of formula (I) encompass any isotopically-labeled (or “radio-labelled”) derivatives of a compound of formula (I). Such a derivative is a derivative of a compound of formula (I) wherein one or more atoms are replaced by an atom having an atomic mass or mass number different from the atomic mass or mass number typically found in nature. Examples of radionuclides that may be incorporated include ²H (also written as “D” for deuterium). As such, in one embodiment, a compound of formula (I) is provided where one or more hydrogen atoms are replaced by one or more deuterium atoms; and the deuterated compound is used in one of the methods provided herein for treating an ANCA associated vasculitis, such as GPA and/or MPA.

In a further embodiment, the compounds of formula (I) may be administered in the form of a prodrug which is broken down in the human or animal body to give a compound of the formula (I). Examples of prodrugs include in vivo hydrolysable esters of a compound of the formula (I).

An in vivo hydrolysable (or cleavable) ester of a compound of the formula (I) that contains a carboxy or a hydroxy group is, for example, a pharmaceutically acceptable ester which is hydrolyzed in the human or animal body to produce the parent acid or alcohol. For examples of ester prodrugs derivatives, see: Curr. Drug. Metab. 2003, 4, 461, incorporated by reference herein in its entirety for all purposes.

Various other forms of prodrugs are known in the art and can be used in the methods provided herein. For examples of prodrug derivatives, see: Nature Reviews Drug Discovery 2008, 7, 255, the disclosure of which is incorporated by reference herein in its entirety for all purposes.

### EXAMPLES

The present invention is further illustrated by reference to the following Examples. However, it should be noted that these Examples, like the embodiments described above, are illustrative and are not to be construed as restricting the scope of the invention in any way.

### Example 1

### INS1007 Reduces PR3 Expression and Neutrophil Serine Protease Activities in Cultured Human Neutrophils

To evaluate the pharmacological effects of INS1007 on surface PR3 expression, total PR3 expression, and NSP activity, stem cells were differentiated to neutrophils in the presence of INS1007 at increasing concentrations. Primary bone marrow (BM) or umbilical cord blood (UC) derived CD34⁺ neutrophil progenitor cells (AllCells; Alameda, Calif.) were cultured for 7 days in STEMSPAN™ SFEM media (Stemcell Technologies; Tukwila, WA) supplemented with recombinant human Stem Cell Factor (Peprotech; Rocky Hill, NJ) and recombinant human IL-3 (Peprotech). The cells were differentiated in culture for another 7 days in STEMSPAN™ SFEM media (Stemcell Technologies) with recombinant human Granulocyte Colony Stimulating Factor (Peprotech), plus increasing concentrations of INS1007 (0-10 μM). At the end of the differentiation/treatment period, cells were harvested for analysis.

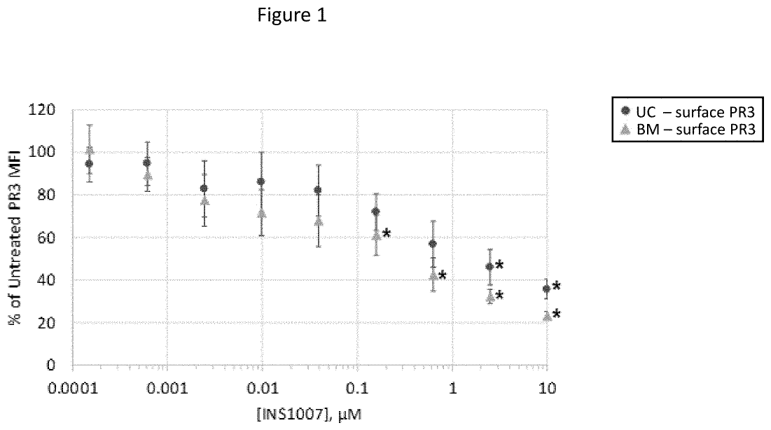

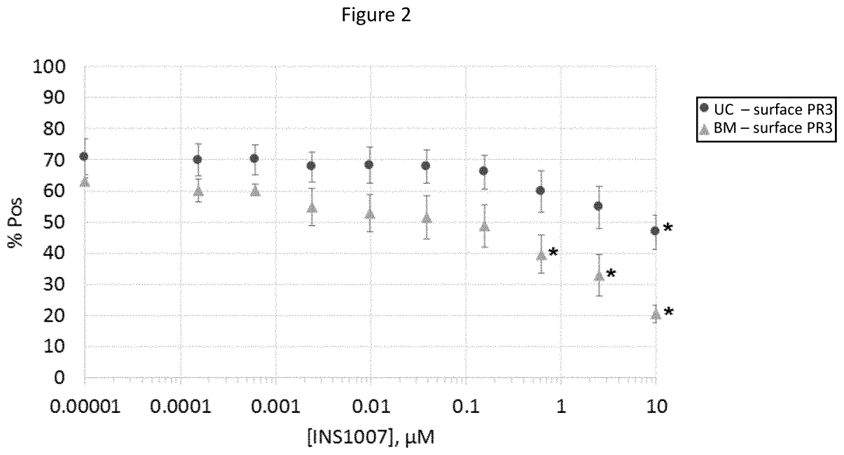

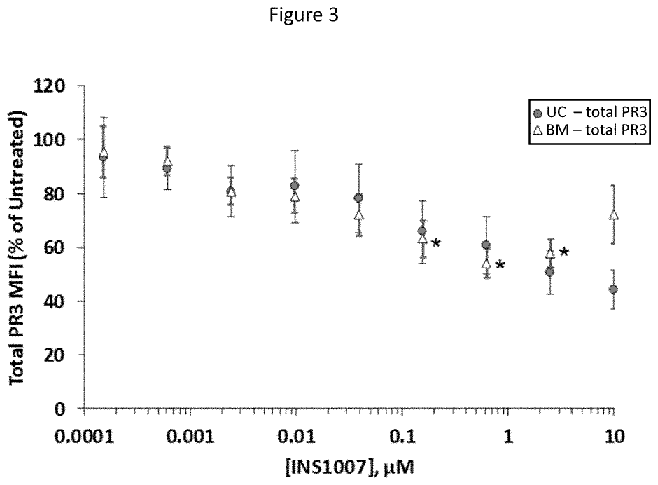

Differentiated neutrophils were harvested for flow cytometry to quantify PR3 expression on the surface of neutrophils (FIGS. 1 and 2) and neutrophil total PR3 expression (FIG. 3).

### Surface PR3 Expression

After harvest, the cells were resuspended in FACS staining buffer and Fc receptors were blocked for 30 min at 4° C. Cells were stained for 30 min with a mouse anti-PR3 monoclonal antibody (Abcam; Cambridge, MA) or a mouse monoclonal IgG isotype control (Abcam), followed by a Cy5-conjugated goat anti-mouse IgG secondary antibody (Abcam), and then analyzed using a GUAVA® EASYCYTE™ 6HT flow cytometer (Millipore; Billerica, Mass.).

### Total PR3 Expression

A spared portion of the cells was further fixed and permeabilized for intracellular staining, with the same anti-PR3 antibody or isotype antibody, followed by the same Cy5-conjugated goat anti-mouse IgG secondary antibody, and then analyzed for total PR3 expression, exhibited by both surface and intracellular PR3 simultaneously.

### NSP Activity Measurements

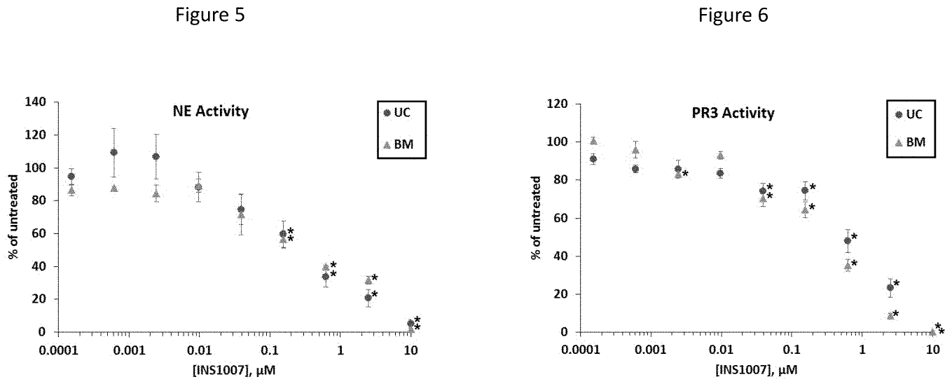

Neutrophils in a separate aliquot of harvested neutrophils were lysed with 10% (v/v) Triton X-100 in PBS and the lysates were added to 96-well plates. Peptide substrates for NE (N-Methoxysuccinyl-Ala-Ala-Pro-Val-7-amido-4-methylcoumarin; Sigma-Aldrich; St. Louis, Mo.), for PR3 (aminobenzoyl-Val-Ala-Asp-Cys-Ala-Asp-Gln-Lys(2,4-dinitrophenyl); GenScript; Piscataway, N.J.), and for CatG (N-Succinyl-Ala-Ala-Pro-Phe p-nitroanilide; Sigma) were added, and either fluorescence or absorbance was quantified using a SYNERGY™ microplate reader (BioTek; Winooski, Vt.). NSP inhibitors were used to test the specificity of enzymatic assays, including elastase inhibitor (Abcam) for NE, sivelestat (Abcam) for PR3, and cathepsin G inhibitor I (Cayman Chemical; Ann Arbor, Mich.) for CatG. A portion of cell lysate samples was retained for protein quantification using a PIERCE™ BCA Protein Assay Kit (Thermo Fisher) according to the manufacturer's instructions. The NSP activities were normalized to cell lysate protein concentrations and the final data were fit with nonlinear, four-parameter, logistical regression analysis by using GRAPHPAD PRISM® software (GraphPad Software Inc.; La Jolla, Calif.). The results are provided in FIGS. 5 and 6 and discussed below.

### Fluorescence Microscopy

In a separate experiment, neutrophils were harvested, stained for 30 min. with either a mouse anti-PR3 monoclonal antibody (Abcam; Cambridge, Mass.) or a mouse monoclonal IgG isotype control (Abcam), followed by a Cy5-conjugated goat anti-mouse IgG secondary antibody (Abcam), fixed in 4% formaldehyde, and mounted to glass slides with PROLONGTM Diamond Antifade Mountant with DAPI (Thermo Fisher; Waltham, Mass.). Samples were imaged with an Axio fluorescence microscope (Zeiss; Oberkochen, Germany) using constant settings while imaging samples from different treatment conditions.

### Results

The results of the fluorescence microscopy experiments demonstrated that neutrophils derived from either bone marrow or umbilical cord stem cells exhibited robust surface expression of PR3. Differentiation in the presence of 10 μM INS1007 markedly reduced surface PR3 in both cell types.

Flow cytometry was employed to quantify mean fluorescence intensity (MFI) associated with surface PR3 expression in cells differentiated in the presence of INS1007 at various concentrations. Following normalization as percent of untreated cells, INS1007 concentration-dependently reduced surface PR3 expression in neutrophils derived from both bone marrow and umbilical cord blood stem cells, with maximum reductions greater than 60% (FIG. 1). In addition, flow cytometry was used to evaluate the percentage of cells without surface PR3 expression. It was found that approximately 30-40% of untreated neutrophils expressed no detectable PR3 on their surface (FIG. 2). INS1007 concentration-dependently increased the percentage of cells with no detectable surface PR3 expression to more than 50% of neutrophils derived from umbilical cord blood stem cells and nearly 80% of neutrophils derived from bone marrow stem cells (FIG. 2).

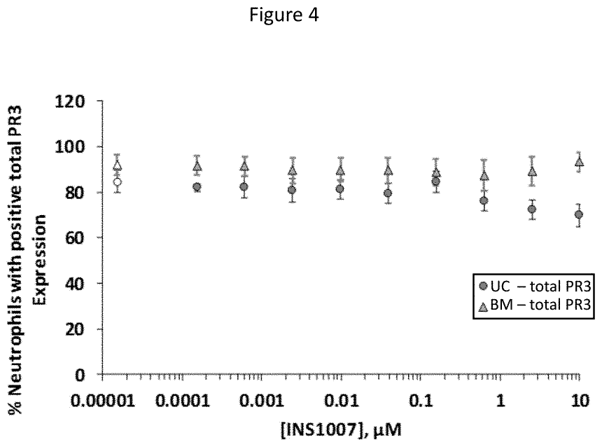

Total PR3 levels did not reach statistically significant reduction with INS1007 in umbilical cord blood stem cell-derived neutrophils, but were significantly reduced in bone marrow stem cell-derived neutrophils, albeit not in a completely concentration-dependent manner (FIG. 3). In addition, flow cytometry was used to evaluate the percentage of cells without total PR3 expression. It was found that approximately 10-15% of untreated neutrophils expressed no detectable total PR3 (FIG. 4). INS1007 did not reduce the percentage of cells with positive total PR3 expression in neutrophils derived from either umbilical cord blood stem cells or bone marrow stem cells (FIG. 4).

INS1007 effects on neutrophil serine protease (NSP) activities within the differentiated neutrophils were evaluated. INS 1007 concentration-dependently decreased the activities of NE and PR3>90%, and >90%, respectively, at the highest concentration tested (10 μM; FIGS. 5-6). In addition, INS1007 concentration dependently descreased the activity of CatG by greater than 75 percent, at the highest concentration tested (10 μM) (data not shown).

Overall, the data show that INS1007 effectively decreases surface PR3 expression and NSP activities during neutrophil differentiation in vitro. Without wishing to be bound by theory, because autoantibodies in patients with GPA bind to surface PR3 to stimulate NSP release and subsequent tissue damage, the pharmacological effects of INS1007 on neutrophils may treat GPA by two distinct mechanisms of action: (i) reduced surface PR3 to decrease the antigen for autoantibody binding; and, (ii) reduced activities of NSP to decrease the tissue damage following neutrophil degranulation.

Moreover, without wishing to be bound by theory, because autoantibodies in patients with MPA bind to surface PR3 and/or to stimulate NSP release and subsequent tissue damage, the pharmacological effects of INS 1007 on neutrophils may treat MPA and other myeloperoxidase (MPO)-ANCA related disorders by reducing activities of NSP to decrease the tissue damage following neutrophil degranulation.

### Example 2

### Bone Marrow Neutrophil Elastase Activity in C57BL/6 Mice Dosed with INS1007

C57BL/6 mice were orally dosed with INS1007, and bone marrow NE activity was tested. Dosing was twice daily for seven days with the following treatment groups. Dosages were split in half to arrive at the toal dose.

| | | Total | | |
|---|---|---|---|---|
| | # | Dose | Treatment | Treatment |
| Treatment | Mice/group | (mg/kg) | Regimen | Duration (Days) |
| Test | 6 | 0.2 | BID | 7 |
| | | | (8AM & 4PM) | |
| Test | 6 | 2 | BID | 7 |
| | | | (8AM & 4PM) | |
| Test | 6 | 20 | BID | 7 |
| | | | (8AM & 4PM) | |

After treatment, all animals had both femurs and tibias removed and bone marrow was aspirated with ice-cold RPMI buffer. Cell pellets were collected after red blood cell lysis and washed with PBS. Ice-cold PBS-Triton X-100 lysis buffer was added to the cell pellet to obtain cell lysate after maximum speed centrifugation.

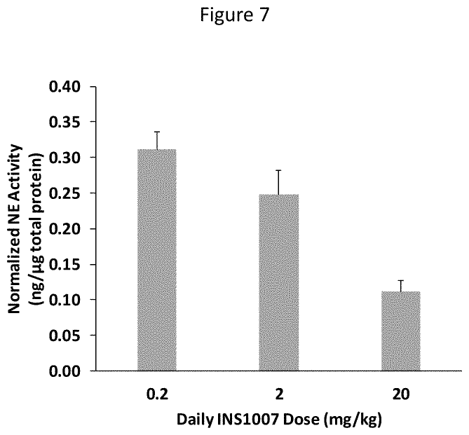

Cell lysates were stored at −80° C. until enzymatic assays were performed. For neutrophil elastase (NE) activity assay, bone marrow cell lysates were added to a 96-well black plate and incubated for 15 minutes in the presence or absence of NE inhibitor (Abcam, Cat # ab142154). A serial dilution of natural mouse elastase protein (Abcam, Cat # ab95133) was used for establishing the standard curve. The NE substrate (Methoxysuccinyl-ala-ala-pro-val-AMC; Sigma, Cat # M9771) was eventually added to the samples. The resulting fluorescence was immediately measured at 350 nm excitation and 450 nm emission, in a kinetic mode by reading every 5 minutes for up to 3 hours at 37° C. The reaction was read on a BIOTEK® SYNERGY™ plate reader. The reaction rate was calculated with the initial slopes of the linear range and a standard curve for the natural mouse elastase was plotted against their slopes. The net NE activity (in the unit of natural mouse elastase amount) was calculated based on the stand curve by subtracting the NE inhibitor control wells from non-inhibited wells. The results are provided in FIG. 7.

### Example 3

### Maintenance of GPA Remission with INS1007

This example describes a randomized, double-blind, placebo-controlled, parallel-group, and multi-center phase 2b trial to assess the efficacy and safety of INS1007 in maintaining GPA remission. Eligible patients are those who have newly diagnosed or relapsing GPA in complete remission. Eligible patients are PR3-ANCA-positive at diagnosis or during the course of their disease. Remission in eligible patients is defined as a BVAS/WG of 0 and symptom-free for at least 30 days, or for at least 90 days at screening. Specifically, at screening, patients are in remission (defined as having a BVAS/WG of 0) and symptom-free for at least 30 days, or for at least 90 days if remission is induced with rituximab. At the time of enrollment, patients can be treated with rituximab, cyclophosphamide, or a steroid. At randomization, patients will stop such treatments.

A screening period of up to 6 weeks per patient will take place. During the screening period, the patient's demographic information, medical history, and smoking history will be obtained, vital signs, ANCA status, and biomarkers will be collected. Clinical laboratory tests, and pregnancy tests will be conducted during this period. Patients who meet all the inclusion criteria will be enrolled in the study. Patients that have other vasculitis (primary or secondary), are pregnant or breastfeeding, will be excluded from the study.

In the treatment arm, patients in GPA remission will be orally administered 25 mg or 40 mg INS1007 once daily for 52 weeks. In the control arm, patients in GPA remission will be orally administered a placebo once daily for 52 weeks. Patients will cease other maintenance medications for GPA once they are randomized to study treatment. Randomization will be stratified based on whether or not the patient was on rituximab maintenance therapy prior to study treatment.

The efficacy of the INS1007 treatment is measured by the following clinical endpoints: the appearance of a GPA relapse in a patient. The relapse can be a major relapse or a minor replapse. A major relapse is defined as reappearance or worsening of disease with (A) a BVAS/WG>0 and involvement of at least one major organ, (B) a life-threatening manifestation, or (C) both (A) and (B). A minor relapse is defined as reappearance or worsening of disease with a BVAS/WG>0, not corresponding to a major relapse, but requiring mild treatment intensification. The number of patients without a relapse will be compared between each arm of the study. The number of patients experiencing a major relapse will be compared between each arm of the study. The number of patients experiencing a minor relapse will be compared between each arm of the study.

In addition, the amount of time to relapse will be measured during the 52-week treatment period and compared between each arm of the study. The number of patients in each arm experiencing a disease flare (BVAS/WG score≥1), and when the respective flare occurs will be quantified. From this information, the rate of flares during the 52-week treatment period can be compared between each arm of the study.

Other efficacy measurements will include (i) the measurement of VDI from baseline to Week 52 for the treatment arms as compared to the placebo control group; (ii) systemic corticosteroid use based on total oral corticosteroid dose and duration of oral corticosteroid use; (iii) quality of life measured by, e.g., a SF-36 score, over the 52-week treatment period compared between groups; and (iv) proteinase 3 neutrophil cell surface expression and/or activity, cathepsin G activity, and/or NE activity in the blood or sputum as compared between groups.

All, documents, patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1. A method for treating an antineutrophil cytoplasmic autoantibody (ANCA) associated vasculitis in a patient in need of treatment, comprising, administering to the patient a pharmaceutical composition comprising an effective amount of a compound of formula (I), or a pharmaceutically acceptable salt thereof, 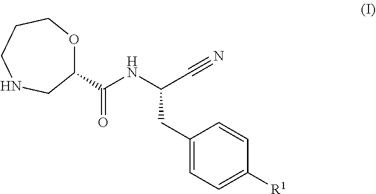
wherein,
R¹ is 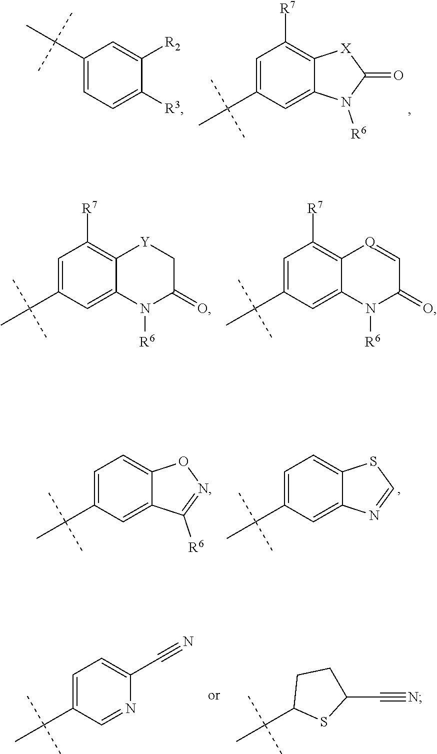
R² is hydrogen, F, Cl, Br, OSO₂C₁₋₃alkyl, or C₁₋₃alkyl;
R³ is hydrogen, F, Cl, Br, CN, CF₃, SO₂C₁₋₃alkyl, CONH₂ or SO₂NR⁴R⁵, wherein R⁴ and R⁵ together with the nitrogen atom to which they are attached form an azetidine, pyrrolidine or piperidine ring; or
R⁶ is C₁₋₃alkyl, optionally substituted by 1, 2 or 3 F and/or optionally by OH, OC₁₋₃alkyl, N(C₁₋₃alkyl)₂, cyclopropyl, or tetrahydropyran;
R⁷ is hydrogen, F, Cl or CH₃;
X is O, S or CF₂;
Y is O or S; and
Q is CH or N.

2.-173. (canceled)

174. The method of claim 1, wherein
R¹ is

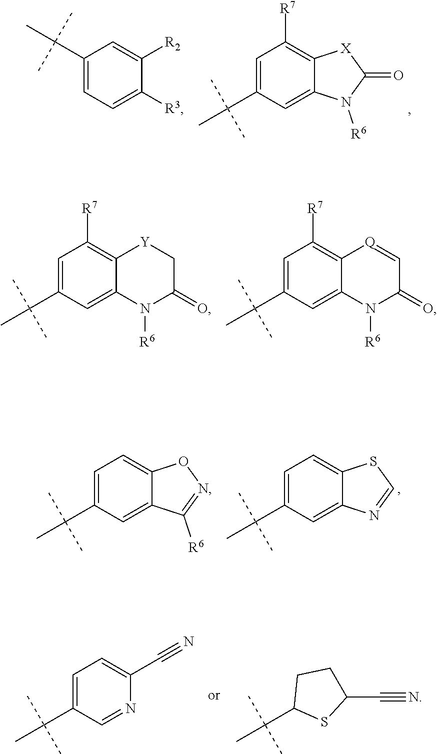

175. The method of claim 1, wherein, 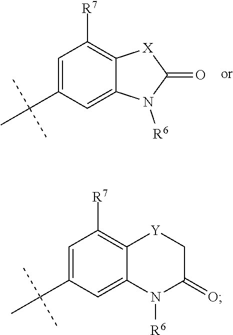
R¹ is
X is selected from O, S or CF₂;
Y is selected from O or S;
R⁶ is selected from C₁₋₃alkyl, wherein said C₁₋₃alkyl is optionally substituted by 1, 2 or 3 F and optionally by one substituent selected from OH, OC₁₋₃alkyl, N(C₁₋₃alkyl)₂, cyclopropyl, or tetrahydropyran;
R⁷ is selected from hydrogen, F, Cl or CH₃;
or a pharmaceutically-acceptable salt thereof.

176. The method of claim 1, wherein R¹ is

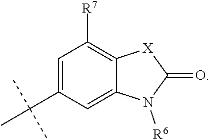

177. The method of claim 1, wherein,
R¹ is 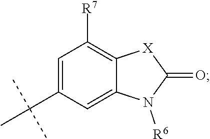
X is O, S or CF₂;
R⁶ is C₁₋₃alkyl, wherein said C₁₋₃alkyl is optionally substituted by 1, 2 or 3 F; and
R⁷ is hydrogen, F, Cl or CH₃.

178. The method of claim 177, wherein X is O and R⁷ is hydrogen.

179. The method of claim 178, wherein R⁶ is methyl or ethyl.

180. The method of claim 1, wherein the compound of Formula (I) is 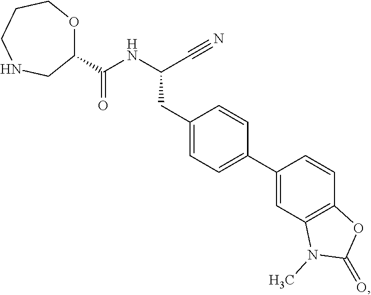 [SMILES: Cn1c(=O)oc2ccc(-c3ccc(C[C@@H](C#N)NC(=O)[C@@H]4CNCCCO4)cc3)cc21]
or a pharmaceutically acceptable salt thereof.

181. The method of claim 1, wherein the administering comprises oral adminsitration.

182. The method of claim 1, wherein the ANCA associated vasculitis is granulomatosis with polyangiitis (GPA).

183. The method of claim 1, wherein the ANCA associated vasculitis is microscopic polyangiitis (MPA).

184. The method of claim 1, wherein the treating comprises decreasing the patient's antineutrophil cytoplasmic autoantibodies (ANCA) blood concentration, as compared to the patient's ANCA blood concentration, prior to treatment.

185. The method of claim 1, wherein the decreasing the ANCA blood concentration of the patient is by at least about 10%, at least about 20%, at least about 25%, at least about 30%, at least about 40%, at least about 50%, at least about 60%, at least about 70% or at least about 80%.

186. The method of claim 185, wherein the ANCA blood concentration is measured in the patient's blood plasma, blood serum or a combination thereof.

187. The method of claim 182, wherein the patient has a Birmingham Vasculitis Activity Score specific for Wegener's granulomatosis (BVAS/WG)>0 at the onset of the treating, and the treating comprises decreasing the BVAS/WG score for the patient, as compared to the BVAS/WG score of the patient prior to the treatment.

188. The method of claim 182, wherein the patient is in GPA remission at the onset of treatment.

189. The method of claim 187, wherein the treating comprises decreasing the BVAS/WG score for the patient by 1 point or more.

190. The method of claim 187, wherein the treating comprises inhibiting a GPA flare, wherein a flare is defined as an increase in the BVAS/WG score of one point or more.

191. The method of claim 1, wherein the treating comprises improving the short form health survey questionnaire (SF-36) score for the patient, as compared to the SF-36 score of the patient prior to the treatment.

192. The method of claim 1, wherein the treating comprises decreasing the number of CD19+ B-cells in the patient, as compared to the number of CD19+ B-cells in the patient prior to the treatment.
